(19) United States
(12) Patent Application Publication
(10) Pub. No.: US 2025/0049800 A1
(43) Pub. Date: Feb. 13, 2025

(54) VCP/p97 INHIBITOR FOR THE TREATMENT OF CANCER

(71) Applicant: CASI Pharmaceuticals, Inc., Rockville, MD (US)

(72) Inventors: Monic Jain Stuart, San Francisco, CA (US); Ronan Le Moigne, San Francisco, CA (US); Daniel James Anderson, EI Cerrito, CA (US); Mark Rolfe, San Francisco, CA (US); Kanya Lakshmi Rajangam, San Francisco, CA (US); Stevan Nicholas Djakovic, San Francisco, CA (US); Jesse Vargas, San Francisco, CA (US); John Gutheil, La Jolla, CA (US); Scott Harris, San Francisco, CA (US)

(21) Appl. No.: 18/709,284

(22) PCT Filed: Nov. 9, 2022
(86) PCT No.: PCT/US2022/049456

§ 371 Date: May 10, 2024

Related U.S. Application Data
(60) Provisional application No. 63/277,725, filed on Nov. 10, 2021.

(51) Int. Cl. A61K 31/519; A61K 31/454; A61K 31/497; A61K 31/502; A61K 31/5025; A61K 31/69; A61K 31/706; A61K 31/7068; A61K 45/06; A61P 35/00; A61P 35/02

(52) U.S. Cl. CPC A61K 31/519; A61K 31/454; A61K 31/497; A61K 31/502; A61K 31/5025; A61K 31/69; A61K 31/706; A61K 31/7068; A61K 45/06; A61P 35/00; A61P 35/02

(57) ABSTRACT

Described herein are methods of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, whereby the subject experiences a therapeutic response.

### CROSS-REFERENCE

This application claims benefit of U.S. Provisional Application No. 63/277,725, filed on Nov. 10, 2021, which is herein incorporated by reference in its entirety.

### BACKGROUND

The Valosin containing protein VCP/p97 and its functions are essential for continued cellular viability.

### SUMMARY OF THE INVENTION

1-(4-(Benzylamino)-5,6,7,8-tetrahydropyrido[2,3-d]pyrimidin-2-yl)-2-methyl-1H-indole-4-carboxamide is a VCP/p97 inhibitor. In one aspect, described herein is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, whereby the subject experiences a therapeutic response. In some embodiments, the pharmaceutical composition comprises a tosylate salt of 1-(4-(benzylamino)-5,6,7,8-tetrahydropyrido[2,3-d]pyrimidin-2-yl)-2-methyl-1H-indole-4-carboxamide. In some embodiments, the dose administered over a twenty-four hour period is between about 450 mg to about 1250 mg, about 450 mg to about 1000 mg, about 450 mg to about 900 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 600 mg to about 1250 mg, about 600 mg to about 1000 mg, about 600 mg to about 900 mg, about 600 mg to about 850 mg, about 600 mg to about 800 mg, about 600 mg to about 750 mg, about 600 mg to about 700 mg, about 900 mg to about 950 mg, about 900 mg to about 1000 mg, about 900 mg to about 1250 mg. In some embodiments, the dose administered over a twenty-four hour period is about 450 mg, 500 mg, 550 mg, 600 mg, 650 mg, 700 mg, 750 mg, 800 mg, 850 mg, 900 mg, 950 mg, 1000 mg or 1250 mg. In some embodiments, the dose administered over a twenty-four hour period is between about 450 mg to about 1250 mg, about 450 mg to about 1000 mg, about 450 mg to about 900 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 600 mg to about 1250 mg, about 600 mg to about 1000 mg, about 600 mg to about 900 mg, about 600 mg to about 850 mg, about 600 mg to about 800 mg, about 600 mg to about 750 mg, about 600 mg to about 700 mg, about 900 mg to about 950 mg, about 900 mg to about 1000 mg, about 900 mg to about 1250 mg, about 1000 mg to about 1250 mg, about 1000 mg to about 1500 mg, or about 1250 mg to about 1500 mg. In some embodiments, the dose administered over a twenty-four hour period is about 450 mg, 500 mg, 550 mg, 600 mg, 650 mg, 700 mg, 750 mg, 800 mg, 850 mg, 900 mg, 950 mg, 1000 mg, 1250 mg, or 1500 mg.

In some embodiments, the dose over a twenty-four hour period is administered as a once daily dose on the days of administration.

In some embodiments, the dose over a twenty-four hour period is administered as a twice daily dose on the days of administration, such that the total dose is divided into two lesser doses. In some embodiments, the dose over a twenty-four hour period is administered as a twice daily dose on the days of administration, wherein the total dose is divided into two lesser doses, and wherein the two lesser doses each contain the same dose. In some embodiments, the dose over a twenty-four hour period is administered as a twice daily dose on the days of administration, wherein the total dose is divided into two lesser doses, and wherein the two lesser doses each contain a different dose. In some embodiments, the dose over a twenty-four hour period is administered as a twice daily dose on the days of administration, wherein the total dose is divided into two lesser doses, and wherein the time interval between the administration of the first daily dose and the administration of the second daily dose is 8 to 12 hours.

In some embodiments, the cancer is selected from the group consisting of a solid tumor, a metastatic form of a solid tumor, an advanced metastatic solid tumor, a lymphoma and an advanced lymphoma. In some embodiments, the cancer is a hematological cancer. In some embodiments, the cancer is selected from the group consisting of acute myeloid leukemia (AML), myelodysplastic syndrome (MDS), myelodysplastic/myeloproliferative overlap neoplasms (MDS/MPN), CMML (chronic myelomonocytic leukemia), atypical CML (chronic myeloid leukemia), multiple myeloma, myeloma, amyloidosis, Waldenstrom's macroglobulinemia (also known as lymphoplasmacytic lymphoma), acute lymphoblastic leukemia (ALL), B-lymphoblastic leukemia, T-lymphoblastic leukemia, lymphoma, B-cell acute lymphoblastic leukemia, T-cell acute lymphoblastic leukemia, B-cell acute lymphoblastic lymphoma, T-cell acute lymphoblastic lymphoma, Burkitt's leukemia/lymphoma, Non-Hodgkin's lymphoma (NHL), chronic lymphocytic leukemia (CLL), small lymphocytic lymphoma (SLL), B-cell NHL, follicular lymphoma, marginal zone lymphoma, mantle cell lymphoma, diffuse large B-cell lymphoma (DLBCL), double/triple hit B-cell lymphoma, myeloproliferative neoplasm (MPN), essential thrombocythemia (ET), polycythemia vera (PV), myelofibrosis, primary myelofibrosis, post-PV myelofibrosis, Post-ET myelofibrosis, chronic myeloid leukemia (CMIL), blastic plasmacytoid dendritic cell neoplasm (BPDCN), M3 AML, and APL (acute promyelocytic leukemia). In some embodiments, the cancer is acute myeloid leukemia (AML) or myelodysplastic syndrome (MDS). In some embodiments, the AML is relapsed AML, recurrent AML, refractory AML or any combination thereof. In some embodiments, the AML is de novo AML, secondary AML including therapy related AML and AML with myelodysplasia-related changes (AML with MRC), biphenotypic acute leukemia (also referred to as Acute Leukemia of ambiguous lineage), or AML with recurrent abnormalities. In some embodiments, the AML is AML with an actionable mutation. In some embodiments, the AML is AML without an actionable mutation. In some embodiments, the MDS is relapsed or refractory MDS. In some embodiments, the MDS is classified by the Revised International Prognostic Scoring System (IPSS-R) as low risk MDS, intermediate risk MDS, high risk MDS, or very high risk MDS. In some embodiments, the MDS is selected from the group consisting of MDS with single lineage dysplasia (MDS-SLD), MDS with multilineage dysplasia (MDS-MLD), MDS with ringed sideroblasts (MDS-RS), MDS with Ringed Sideroblasts with single lineage dysplasia (MDS-RS-SLD), MDS with Ringed Sideroblasts (MDS-RS), MDS with Ringed Sideroblasts with multilineage dysplasia (MDS-RS-HLD), MDS with excess blasts 1 and/or 2 (MDS-EB-1, MDS-EB-2), MDS unclassifiable (MDS-U), and MDS with isolated del (5q).

In some embodiments, the subject is treated irrespective of the subject's mutation or cytogenetic status.

In some embodiments, therapeutic response comprises a complete remission, complete remission without minimal residual disease, complete remission with incomplete hematologic recovery, morphologic leukemia-free state or partial remission, hematological improvement, complete cytogenetic response, transfusion independence, red blood cell transfusion independence or platelet transfusion independence, or eligibility for stem cell transplantation.

In some embodiments, therapeutic response comprises an increase in overall survival, an increase in relapse free survival, an increase in event free survival, an increased duration of response or a reduction in cumulative incidence of relapse.

In some embodiments, the pharmaceutical composition is administered in a regimen comprising (a) 4 sequential days of administering the drug to a subject followed by 3 sequential days of no administration, (b) 5 sequential days of administering the drug to a subject followed by 2 sequential days of no administration, (c) a once weekly dosage, or (d) a twice-weekly dosage. In some embodiments, the administration regimen is repeated. In some embodiments, the pharmaceutical composition is administered in a 28 day cycle comprising administration on days 1-4, 8-11, 15-18, and 22-25 of each cycle. In some embodiments, the 28 day cycle is repeated at least once. In some embodiments, the 28 day cycle is repeated until relapse.

In some embodiments, the pharmaceutical composition is administered orally.

In some embodiments, the pharmaceutical composition is administered as a tablet or a capsule.

In some embodiments, the cancer is AML and the subject carries one or more mutations in a locus selected from the group consisting of ABL1, ASXL1, BCOR, BCORL1, BCR, BRAF, CALR, CBFB, CBL, CBLB, CDKN2A, CEBPA, CSF3R, CUX1, DEK, DNMT3A, ETV6, EZH2, FBXW7, FLT3, GATA1, GATA2, GNAS, HRAS, IDH1, IDH2, IKZF1, JAK2, JAK3, KDM6A, KIT, KMT2A, MECOM (EVI1), MLL, MLLT3, MPL, MYD88, MYH11, NOTCH1, NPM1, NUP214, NRAS, PDGFRA, PHF6, PTEN, PTPN11, RAD21, RUNX1, SF3B1, SRSF2, SMC1A, SMC3, STAG2, TET2, TP53, U2AF1, WT1, and ZRSR2.

In some embodiments, the treatment further includes administration of a second therapeutic agent. In some embodiments, the second therapeutic agent is a DNA damaging agent, a hypomethylating agent, an agent that interferes with DNA synthesis or an agent that interferes with DNA replication. In some embodiments, the second therapeutic agent is decitabine, azacytidine, or cytarabine. In some embodiments, the second therapeutic agent is cytarabine dosed in a 7+3 regimen with an anthracycline antibiotic. In some embodiments, the 7+3 comprises 7 days of cytarabine and 3 days of an anthracycline antibiotic selected from daunorubicin, doxorubicin, idarubicin, and mitoxantrone. In some embodiments, the second therapeutic agent is a tyrosine kinase inhibitor. In some embodiments, the second therapeutic agent is a DNA damage repair inhibitor. In some embodiments, the second therapeutic agent is an inhibitor of ATM, ATR, PARP, or Chk1. In some embodiments, the second therapeutic agent is a proteasome inhibitor. In some embodiments, the second therapeutic agent is Velcade (bortezomib), Kyprolis (carfilzomib), or Ninlaro (ixazomib). In some embodiments, the second therapeutic agent is lenalidomide, pomalidomide, dexamethasone or a combination of two thereof where one agent is dexamethasone. In some embodiments, the second therapeutic agent is an inhibitor of FLT3, IDH1, or IDH2. In some embodiments, the second therapeutic agent is an immune oncology agent or an immune modulation agent. In some embodiments, the second therapeutic agent is Sarclisa (isatuximab) or other CD38 directed monoclonal antibody. In some embodiments, the anti-CD38 antibody is Sarclisa (isatuximab). In some embodiments, the anti-CD38 antibody is daratumumab. In some embodiments, the second therapeutic agent comprises a menin inhibitor. In some embodiments, the second therapeutic agent comprises SNDX-5613. In some embodiments, the second therapeutic agent comprises KO-539. In some embodiments, the second therapeutic agent comprises a selective inhibitor of nuclear export. In some embodiments, the second therapeutic agent comprises selinexor. In some embodiments, the cancer is selected from the group consisting of a solid tumor, a metastatic form of a solid tumor, an advanced metastatic solid tumor, a lymphoma and an advanced lymphoma. In some embodiments, the subject has undergone at least one prior therapy. In some embodiments, the second therapeutic agent comprises gilteritinib, midostaurin, quizartinib or an analog thereof. In some embodiments, the second therapeutic agent comprises gilteritinib or an analog thereof. In some embodiments, the second therapeutic agent comprises midostaurin or an analog thereof. In some embodiments, the second therapeutic agent comprises quizartinib or an analog thereof. In some embodiments, the cancer comprises a FLT3 mutation. In some embodiments, the second therapeutic agent inhibits poly ADP ribose polymerase (PARP). In some embodiments, the second therapeutic agent comprises talazoparib, olaparib, niraparib or an analog thereof. In some embodiments, the cancer comprises a BRCA-2 mutation. In some embodiments, the cancer is MSI high. In some embodiments, the cancer comprises a mutation that impairs homologous recombination. In some embodiments, the second therapeutic agent inhibits Bcl-2. In some embodiments, the second therapeutic agent comprises a BH3 mimetic. In some embodiments, the BH3 mimetic comprises venetoclax or an analog thereof. In some embodiments, the cancer is a bcr-abl negative myeloid neoplasm. In some embodiments, the administration does not result in a visual impairment of the subject. In some embodiments, the second therapeutic agent is administered prior to the administration of the pharmaceutical composition. In some embodiments, the second therapeutic agent is administered at about 24 hours or 1 day prior to the administration of the pharmaceutical composition.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### BRIEF DESCRIPTION OF THE FIGURES

FIG. 1. Illustrates the plasma concentration-time plots for Compound 1 in cohorts 1-7 doses at 25 mg, 50 mg, 100 mg, 175 mg, 275 mg, 350 mg and 450 mg on Day 1.

FIG. 2. Illustrates the plasma concentration-time plots for Compound 1 in cohorts 1-7 doses at 25 mg, 50 mg, 100 mg, 175 mg, 275 mg, 350 mg and 450 mg on Day 4.

FIGS. 3A-3D. Illustrates the AUC₀₋ₜ vs dose for Day 1 and Day 4 (FIGS. 3A and 3C) and the Cₘₐₓ vs dose for Day 1 and Day 4 (FIGS. 3B and 3D).

FIG. 4. Illustrates a comparison of the plasma concentration-time plots for Compound 1 and for CB-5083.

FIG. 5. Depicts the structure of 1-(4-(benzylamino)-5,6,7,8-tetrahydropyrido[2,3-d]pyrimidin-2-yl)-2-methyl-1H-indole-4-carboxamide which is CB-5339 and also referred to as Compound 1 herein.

FIG. 6. Illustrates clinical objectives and endpoints for administration of 1-(4-(benzylamino)-5,6,7,8-tetrahydropyrido[2,3-d]pyrimidin-2-yl)-2-methyl-1H-indole-4-carboxamide. Abbreviations used in FIG. 6: AML=acute myeloid leukemia; AUC_{∞}=area under the plasma concentration-time curve from time zero to 24 hours; AUC0-t=area under the concentration-time curve from time zero to the last quantifiable timepoint; Cmax=maximum observed plasma concentration CR=complete remission; CRi=complete remission within complete hematologic recovery; DoCR=duration of complete remission/complete remission with incomplete hematologic recovery; DoR=duration of remission; ELN=European LeukemiaNet; IWG=International Working Group; MTD=maximum tolerated dose; MDS=myelodysplastic syndrome; MPN=Myeloproliferative Neoplasm; PD=pharmacodynamic; PK=pharmacokinetic; PR=partial remission; RP2D=recommended Phase 2 dose; RR=relapsed/refractory; tó=half-life; tmax=time to maximum concentration.

FIG. 7A. Illustrates the AUC₀₋ₜ vs dose for Day 1.

FIG. 7B. Illustrates the AUC₀₋ₜ vs dose for Day 4.

FIG. 8A. Illustrates the Cₘₐₓ vs dose for Day 1.

FIG. 8B. Illustrates the Cₘₐₓ vs dose for Day 4.

FIG. 9. Illustrates a comparison of the plasma concentration-time plots for Compound 1 QD and BID dosing at Day 1.

FIG. 10. Illustrates a comparison of the plasma concentration-time plots for Compound 1 QD and BID dosing at Day 4.

### DETAILED DESCRIPTION OF THE INVENTION

Despite the considerable heterogeneity of tumors, malignant transformation confers common characteristics to cancer cells, the so called “hallmarks of cancer”, which include replicative immortality, resistance to negative growth signals and apoptosis, and the ability to induce angiogenesis. These properties, while providing cancer cells with survival advantages, also expose them to stress conditions that are not ordinarily experienced by normal cells and force them to rely on intracellular pathways that are non-oncogenic, per se, but that nevertheless become essential for their survival (i.e. “non-oncogenic addiction”). Based on this observation, mechanisms of stress overload that interact with cancer cells in a synthetic lethal manner are considered a viable option for therapeutic intervention.

Two major stresses cancer cells face are proteotoxic and genomic stress. Proteotoxic stress, or the inappropriate overproduction of intra- and extra-cellular proteins, including normal proteins, mutant cancer-associated proteins, and novel cancer-associated fusion proteins, drives a dependence on protein homeostasis pathways and the unfolded protein response. The high level of proteotoxic stress in cancer cells suggests that therapeutic strategies targeting protein homeostasis should have notable anticancer activity by inducing apoptosis in those cancer cells that are over-dependent on their protein homeostasis machinery. Although the protein homeostasis and degradation pathways are not specific to cancer cells, the over-dependence of cancer cells on these systems may sensitize them to specific inhibitors of protein homeostasis and result in anticancer activity with less toxicity to normal cells. Genomic stress arises in cancer cells from initial mutations to genes involved in genomic integrity that enables neoplastic transformation and from their rapid growth potential and the DNA replicative errors that accompany that growth, potentially amplified by the number of mutations cancer cells can harbor in the machinery responsible for the fidelity of replication and proper repair of DNA damage as it arises during these processes.

There are two major intracellular degradative pathways that control protein homeostasis—the ubiquitin proteasome system (UPS) and the autophagy lysosome system (ALS). Valosin containing protein VCP/p97, has a well-described role in the UPS where it extracts misfolded proteins from the endoplasmic reticulum in a process termed endoplasmic reticulum-associated degradation (ERAD) and chaperones subsets of proteins to the proteasome for degradation. VCP/p97 also plays a critical role in the regulation of chromatin-related events such as DNA damage response and repair. VCP/p97 is known to be overproduced in multiple cancers. Therefore, pharmacologic interference with VCP/p97 function is expected to have meaningful antitumor effects by generating irresolvable endoplasmic reticulum (ER) stress and/or irresolvable genotoxic stress. Thus, inhibitors of VCP/p97 function could provide a mechanism to exploit a cancer cell's addiction to protein homeostasis and DNA damage repair pathways.

There is a need for inhibitors of VCP/p97 which are useful cancer therapeutics. One such inhibitor is 1-(4-(Benzylamino)-5,6,7,8-tetrahydropyrido[2,3-d]pyrimidin-2-yl)-2-methyl-1H-indole-4-carboxamide (Compound 1). Provided herein are method of treating cancers with Compound 1, including dosing of Compound 1 and administration regimens for achieving a therapeutic response, such as in difficult to treat cancer types and sub-types. The methods herein include methods for enhancing efficacy and/or reducing or mitigating potential side effects. Also provided are methods for enhancing efficacy or therapeutic response and/or increasing the scope of cancers to be treated with combinations of Compound 1 and additional therapeutic agent(s).

### Definitions

As used in the specification and appended claims, unless specified to the contrary, the following terms have the meaning indicated below.

The singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range varies between 1% and 15% of the stated number or numerical range. The term “comprising” (and related terms such as “comprise” or “comprises” or “having” or “including”) is not intended to exclude that which in other certain embodiments, for example, an embodiment of any composition of matter, composition, method, use, or process, or the like, described herein, may “consist of” or “consist essentially of” the described features.

“Optional” or “optionally” means that a subsequently described event or circumstance may or may not occur and that the description includes instances when the event or circumstance occurs and instances in which it does not.

As used herein, “treatment” or “treating” or “palliating” or “ameliorating” are used interchangeably herein. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are administered to a patient at risk of developing a particular disease, or to a patient reporting one or more of the physiological symptoms of a disease, even though a diagnosis of this disease has not been made.

The Compound 1 dosing amounts and ranges described herein refer to the dose of Compound 1 free base or the dose of a pharmaceutically acceptable salt form of Compound 1.

In some embodiments, “participant”, “subject”, and “patient” are used interchangeably. In some embodiments, “subject” refers to a healthy individual. In other embodiments, “subject” refers to a patient in need of treatment. In some embodiments, “subject” refers to a human or an animal, particularly a mammal. In some embodiments, “subject” refers to a human. In some embodiments, “subject” refers to a non-human mammal.

1-(4-(Benzylamino)-5,6,7,8-tetrahydropyrido[2,3-d]pyrimidin-2-yl)-2-methyl-1H-indole-4-carboxamide (Compound 1) is a VCP/p97 inhibitor. 1-(4-(Benzylamino)-5,6,7,8-tetrahydropyrido[2,3-d]pyrimidin-2-yl)-2-methyl-1H-indole-4-carboxamide (Compound 1) is CB-5339. “Compound 1”, “CB-5339”, or “1-(4-(benzylamino)-5,6,7,8-tetrahydropyrido[2,3-d]pyrimidin-2-yl)-2-methyl-1H-indole-4-carboxamide” refers to the compound with the following structure:

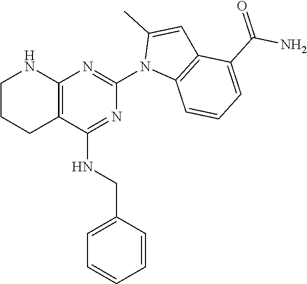 [SMILES: Cc1cc2c(C(N)=O)cccc2n1-c1nc2c(c(NCc3ccccc3)n1)CCCN2]

In some embodiments, Compound 1 is in the form of a pharmaceutically acceptable salt. In some embodiments, Compound 1 is in the form of a pharmaceutically acceptable salt selected from a hydrochloric acid salt, hydrobromic acid salt, sulfuric acid salt, methanesulfonic acid salt, benzenesulfonic acid salt, toluenesulfonic acid salt, phosphoric acid salt, citric acid salt, tartaric acid salt, gentisic acid salt, acetic acid salt, adipic acid salt, benzoic acid salt, glutamic acid salt, glycolic acid salt, lactic acid salt, malic acid salt, malonic acid salt, and succinic acid salt. In some embodiments, Compound 1 is in the form of a toluenesulfonic acid salt. In some embodiments, Compound 1 is in the form of a sulfuric acid salt. In some embodiments, Compound 1 is in the form of a hydrochloric acid salt. In some embodiments, Compound 1 is a free base. In addition, Compound 1 can exist in unsolvated as well as solvated forms with pharmaceutically acceptable solvents. In some embodiments, Compound 1 is solvated. In some embodiments, Compound 1 is unsolvated.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of therapeutically active agent in combination with a suitable anion, or in alternative embodiments, an anionic form of therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

Solvates contain either stoichiometric or non-stoichiometric amounts of a solvent, and are formed during the process of product formation or isolation with pharmaceutically acceptable solvents such as water, ethanol, methanol, tert-butyl methyl ether (MTBE), diisopropyl ether (DIPE), ethyl acetate, isopropyl acetate, isopropyl alcohol, methyl isobutyl ketone (MIBK), methyl ethyl ketone (MEK), acetone, nitromethane, tetrahydrofuran (THF), dichloromethane (DCM), dioxane, heptanes, toluene, anisole, acetonitrile, and the like. In some embodiments, solvates are formed using, but not limited to, Class 3 solvent(s). In some embodiments, solvates are formed using, but not limited to, Class 2 solvent(s). Categories of solvents are defined in, for example, the International Conference on Harmonization of Technical Requirements for Registration of Pharmaceuticals for Human Use (ICH), “Impurities: Guidelines for Residual Solvents Q3C(R6),” (October 2016). Hydrates are formed when the solvent is water, or alcoholates are formed when the solvent is alcohol.

In some embodiments, Compound 1 is prepared in various forms, including but not limited to, an amorphous phase, crystalline forms, milled forms, and nano-particulate forms.

### Methods

Disclosed herein are methods of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, whereby the subject experiences a therapeutic response. In some embodiments is a method of treating cancer in a subject in need thereof comprising administering to the subject a pharmaceutical composition comprising a pharmaceutically acceptable salt of 1-(4-(benzylamino)-5,6,7,8-tetrahydropyrido[2,3-d]pyrimidin-2-yl)-2-methyl-1H-indole-4-carboxamide at a dose of about 450 mg to about 1500 mg. In some embodiments is a method of treating cancer in a subject in need thereof comprising administering to the subject a pharmaceutical composition comprising a toluenesulfonic acid salt of 1-(4-(benzylamino)-5,6,7,8-tetrahydropyrido[2,3-d]pyrimidin-2-yl)-2-methyl-1H-indole-4-carboxamide at a dose of about 450 mg to about 1500 mg. In some embodiments is a method of treating cancer in a subject in need thereof comprising administering to the subject a pharmaceutical composition comprising a sulfuric acid salt of 1-(4-(benzylamino)-5,6,7,8-tetrahydropyrido[2,3-d]pyrimidin-2-yl)-2-methyl-1H-indole-4-carboxamide at a dose of about 450 mg to about 1500 mg. In some embodiments is a method of treating cancer in a subject in need thereof comprising administering to the subject a pharmaceutical composition comprising a hydrochloric acid salt of 1-(4-(benzylamino)-5,6,7,8-tetrahydropyrido[2,3-d]pyrimidin-2-yl)-2-methyl-1H-indole-4-carboxamide at a dose of about 450 mg to about 1500 mg.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide free base at a dose of about 450 mg to about 1500 mg, whereby the subject experiences a therapeutic response.

In some embodiments a therapeutic response comprises the achievement of one or more response criteria as measured by an established or proposed medical standard, such as by a national or internationally recognized medical consortium. Exemplary standards include 2017 European LeukemiaNet (ELN) response criteria for AML (Dohner et al. (2017), Blood Vol. 129(4) 424-427), the 2006 revised International Working Group (IWG) response criteria for MDS (Cheson et al. (2006), Blood Vol. 108(2), 419-25), the proposal by an international consortium of uniform response criteria for myelodysplastic/myeloproliferative neoplasms (MDS/MPN) in adults (Savona et al. Blood (2015), Vol. 125(12), 1857-65), see also see Tefferi et al. Blood (2013), Vol. 122(8), 1395-98.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, where the dose administered over a twenty-four hour period is between about 450 mg to about 1250 mg, about 450 mg to about 1000 mg, about 450 mg to about 900 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 600 mg to about 1250 mg, about 600 mg to about 1000 mg, about 600 mg to about 900 mg, about 600 mg to about 850 mg, about 600 mg to about 800 mg, about 600 mg to about 750 mg, about 600 mg to about 700 mg, about 900 mg to about 950 mg, about 900 mg to about 1000 mg, about 900 mg to about 1250 mg, whereby the subject experiences a therapeutic response. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, where the dose administered over a twenty-four hour period is between about 450 mg to about 1250 mg, about 450 mg to about 1000 mg, about 450 mg to about 900 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 600 mg to about 1250 mg, about 600 mg to about 1000 mg, about 600 mg to about 900 mg, about 600 mg to about 850 mg, about 600 mg to about 800 mg, about 600 mg to about 750 mg, about 600 mg to about 700 mg, about 900 mg to about 950 mg, about 900 mg to about 1000 mg, about 900 mg to about 1250 mg, about 1000 mg to about 1250 mg, about 1000 mg to about 1500 mg, or about 1250 mg to about 1500 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1250 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1000 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 900 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 850 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 800 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 750 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 700 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 650 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 600 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 1500 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 1250 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 1000 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 900 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 850 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 800 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 750 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 700 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg to about 650 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 900 mg to about 1250 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 900 mg to about 1000 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 1000 mg to about 1500 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 1250 mg to about 1500 mg. In some embodiments, the dose administered over a twenty-four hour period is administered BID (twice daily), such that a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, is administered at a total dose over the twenty-four hour period is between about 450 mg to about 1250 mg, about 450 mg to about 1000 mg, about 450 mg to about 900 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 600 mg to about 1250 mg, about 600 mg to about 1000 mg, about 600 mg to about 900 mg, about 600 mg to about 850 mg, about 600 mg to about 800 mg, about 600 mg to about 750 mg, about 600 mg to about 700 mg, about 900 mg to about 950 mg, about 900 mg to about 1000 mg, about 900 mg to about 1250 mg. In some embodiments, the dose administered over a twenty-four hour period is administered BID (twice daily), such that a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, is administered at a total dose over the twenty-four hour period is between about 450 mg to about 1250 mg, about 450 mg to about 1000 mg, about 450 mg to about 900 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 600 mg to about 1250 mg, about 600 mg to about 1000 mg, about 600 mg to about 900 mg, about 600 mg to about 850 mg, about 600 mg to about 800 mg, about 600 mg to about 750 mg, about 600 mg to about 700 mg, about 900 mg to about 950 mg, about 900 mg to about 1000 mg, about 900 mg to about 1250 mg, about 1000 mg to about 1250 mg, about 1000 mg to about 1500 mg, or about 1250 mg to about 1500 mg. In some embodiments, BID dosing, a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, is administered at each dose at about 225 mg, 250 mg, 275 mg, 300 mg, 350 mg, 400 mg, 450 mg, 500 mg or 600 mg. In some embodiments, each dose of the BID dosing schedule is administered at a timepoint at least 4, 5, 6, 7, 8, 9, 10, 11, 12, 13, 14, 15, or 16 hours apart. In some embodiments, each dose of the BID dosing schedule is administered at a timepoint at least 4, 5, 6, 7, or 8 hours apart. In some embodiments, each dose of the BID dosing schedule is administered at a timepoint at least 9, 10, 11, 12, 13 or 14 hours apart. In some embodiments, each dose of the BID dosing schedule is administered at a timepoint at least 4, 6, 8, 10, 12, 14, or 16 hours apart. In some embodiments, each dose of the BID dosing schedule is administered at a timepoint between 4-16, 4-12, 6-16, 6-14, 6-12, 6-10, 8-14, 8-12, 8-10, 9-10, 9-11, 9-12, 9-13, 9-14, 10-11, 10-12, 10-13, 10-14, 11-12, 11-13, 11-14, 12-13, 12-14,13-14, or 14-16 hours apart. In some embodiments, each dose of the BID dosing schedule is administered at a timepoint between 9-10, 9-11, 9-12, 9-13, 9-14, 10-11, 10-12, 10-13, 10-14, 11-12, 11-13, 11-14, 12-13, 12-14 or 13-14 hours apart. In some embodiments, each dose of the BID dosing schedule is administered at a timepoint between 4-16, 4-12, 6-16, 6-14, 6-12, 6-10, 8-14, 8-12, 8-10, or 14-16 hours apart.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg, 500 mg, 550 mg, 600 mg, 650 mg, 700 mg, 750 mg, 800 mg, 850 mg, 900 mg, 950 mg, 1000 mg or 1250 mg. whereby the subject experiences a therapeutic response. In some embodiments, the dose administered over a twenty-four hour period is administered BID (twice daily), such that a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, is administered at a total dose over the twenty-four hour period is about 450 mg, 500 mg, 550 mg, 600 mg, 650 mg, 700 mg, 750 mg, 800 mg, 850 mg, 900 mg, 950 mg, 1000 mg or 1250 mg. In some embodiments, the dose administered over a twenty-four hour period is administered BID (twice daily), such that a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, is administered at a total dose over the twenty-four hour period is about 450 mg, 500 mg, 550 mg, 600 mg, 650 mg, 700 mg, 750 mg, 800 mg, 850 mg, 900 mg, 950 mg, 1000 mg, 1250 mg, or 1500 mg. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg. In some embodiments, the 450 mg dose is comprised in two doses that are administered over the twenty-four hour period, such as two doses of 225 mg each. In some embodiments, the 450 mg dose is comprised in a single dose administered over the twenty-four hour period. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 500 mg. In some embodiments, the 500 mg dose is comprised in two doses that are administered over the twenty-four hour period, such as two doses of 250 mg each. In some embodiments, the 500 mg dose is comprised in a single dose administered over the twenty-four hour period. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 600 mg. In some embodiments, the 600 mg dose is comprised in two doses that are administered over the twenty-four hour period, such as two doses of 300 mg each. In some embodiments, the 600 mg dose is comprised in a single dose administered over the twenty-four hour period. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 700 mg. In some embodiments, the 700 mg dose is comprised in two doses that are administered over the twenty-four hour period, such as two doses of 350 mg each. In some embodiments, the 700 mg dose is comprised in a single dose administered over the twenty-four hour period. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 800 mg. In some embodiments, the 800 mg dose is comprised in two doses that are administered over the twenty-four hour period, such as two doses of 400 mg each. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 900 mg. In some embodiments, the 900 mg dose is comprised in two doses that are administered over the twenty-four hour period, such as two doses of 450 mg each. In some embodiments, the 900 mg dose is comprised in a single dose administered over the twenty-four hour period. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 1000 mg. In some embodiments, the 1000 mg dose is comprised in two doses that are administered over the twenty-four hour period, such as two doses of 500 mg each. In some embodiments, the 1000 mg dose is comprised in a single dose administered over the twenty-four hour period.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the cancer is selected from the group consisting of a solid tumor, a metastatic form of a solid tumor, an advanced metastatic solid tumor, a lymphoma, and an advanced lymphoma, whereby the subject experiences a therapeutic response. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the cancer is a solid tumor. In some such embodiments, the dose is administered as a single dose or administered BID in two divided doses. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the cancer is a metastatic form of a solid tumor. In some such embodiments, the dose is administered as a single dose or administered BID in two divided doses. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the cancer is an advanced metastatic solid tumor. In some such embodiments, the dose is administered as a single dose or administered BID in two divided doses. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the cancer is a lymphoma. In some such embodiments, the dose is administered as a single dose or administered BID in two divided doses. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the cancer is an advanced lymphoma. In some such embodiments, the dose is administered as a single dose or administered BID in two divided doses.

In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is a bcr-abl negative myeloid neoplasm. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is selected from acute myeloid leukemia (AML), myelodysplastic syndrome (MDS), myelodysplastic/myeloproliferative overlap neoplasms (MDS/MPN), such as MDS/MPN-RS-T, MDS/MPN unclassifiable, CMML (chronic myelomonocytic leukemia), such as CMML-1 and CMML-2, aCML (atypical chronic myeloid leukemia), multiple myeloma, myeloma, amyloidosis, Waldenstrom's macroglobulinemia (also known as lymphoplasmacytic lymphoma), acute lymphoblastic leukemia (ALL), B-lymphoblastic leukemia, T-lymphoblastic leukemia, lymphoma, B-cell acute lymphoblastic leukemia, T-cell acute lymphoblastic leukemia, B-cell acute lymphoblastic lymphoma, T-cell acute lymphoblastic lymphoma, Burkitt's leukemia/lymphoma, Non-Hodgkin's lymphoma (NHL), chronic lymphocytic leukemia (CLL), small lymphocytic lymphoma (SLL), B-cell NHL, follicular lymphoma, marginal zone lymphoma, mantle cell lymphoma, diffuse large B-cell lymphoma (DLBCL), double/triple hit B-cell lymphoma, myeloproliferative neoplasm (MPN), including CES-NOS, and MPN unclassifiable, essential thrombocythemia (ET), polycythemia vera (PV), myelofibrosis, chronic neutrophilic leukemia (CNL), primary myelofibrosis, post-PV myelofibrosis, Post-ET myelofibrosis, post-PV/ET myelofibrosis, myelofibrosis secondary to PV and ET-prognostic model [MYSEC-PM], chronic myeloid leukemia (CML), blastic plasmacytoid dendritic cell neoplasm (BPDCN), M3 AML, and APL (acute promyelocytic leukemia). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is acute myeloid leukemia (AML). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is myelodysplastic syndrome (MDS). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is myelodysplastic/myeloproliferative overlap neoplasms (MDS/MPN). In some aspects, MDS/MPN is MDS/MPN-RS-T or MDS/MPN unclassifiable. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is CMML (chronic myelomonocytic leukemia). In some aspects, CMML is CMML-1 or CMML-2. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is atypical chronic myeloid leukemia (aCML). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is multiple myeloma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is myeloma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is amyloidosis. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is Waldenstrom's macroglobulinemia (also known as lymphoplasmacytic lymphoma). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is acute lymphoblastic leukemia (ALL). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is B-lymphoblastic leukemia. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is T-lymphoblastic leukemia. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is B-cell acute lymphoblastic leukemia. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is T-cell acute lymphoblastic leukemia. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, wherein the hematological cancer is B-cell acute lymphoblastic lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is T-cell acute lymphoblastic lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is Burkitt's leukemia/lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is Non-Hodgkin's lymphoma (NHL). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is chronic lymphocytic leukemia (CLL). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, wherein the hematological cancer is small lymphocytic lymphoma (SLL). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is B-cell NHL. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is follicular lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is marginal zone lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is mantle cell lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is diffuse large B cell lymphoma (DLBCL). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is double/triple hit B cell lymphoma. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is myeloproliferative neoplasm (MPN). In some aspects, MPN is CES-NOS or MPN unclassifiable. In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is essential thrombocythemia (ET). In some such embodiments, the dose is administered as a single dose or administered BID in two divided doses. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is polycythemia vera (PV). In some such embodiments, the dose is administered as a single dose or administered BID in two divided doses.

In some embodiments of the methods described herein, the dose is administered as a single dose. In some embodiments of the methods described herein, the dose is administered BID in two divided doses. In some embodiments of the methods described herein, the dose is administered BID in two divided doses wherein the two divided doses each contain the same dose. In some embodiments of the methods described herein, the dose is administered BID in two divided doses wherein the two divided doses each contain a different dose. In some embodiments of the methods described herein, the dose is administered BID in two divided doses wherein the time interval between the administration of the first dose and the administration of the second dose is 4 to 16 hours. In some embodiments of the methods described herein, the dose is administered BID in two divided doses wherein the time interval between the administration of the first dose and the administration of the second dose is 6 to 16 hours. In some embodiments of the methods described herein, the dose is administered BID in two divided doses wherein the time interval between the administration of the first dose and the administration of the second dose is 4 to 14 hours. In some embodiments of the methods described herein, the dose is administered BID in two divided doses wherein the time interval between the administration of the first dose and the administration of the second dose is 6 to 14 hours. In some embodiments of the methods described herein, the dose is administered BID in two divided doses wherein the time interval between the administration of the first dose and the administration of the second dose is 4 to 12 hours. In some embodiments of the methods described herein, the dose is administered BID in two divided doses wherein the time interval between the administration of the first dose and the administration of the second dose is 6 to 12 hours. In some embodiments of the methods described herein, the dose is administered BID in two divided doses wherein the time interval between the administration of the first dose and the administration of the second dose is 8 to 12 hours. In some embodiments of the methods described herein, the dose is administered BID in two divided doses wherein the time interval between the administration of the first dose and the administration of the second dose is 6 to 8 hours. In some embodiments of the methods described herein, the dose is administered BID in two divided doses wherein the time interval between the administration of the first dose and the administration of the second dose is 8 to 10 hours. In some embodiments of the methods described herein, the dose is administered BID in two divided doses wherein the time interval between the administration of the first dose and the administration of the second dose is 10 to 12 hours. In some embodiments of the methods described herein, the dose is administered BID in two divided doses wherein the time interval between the administration of the first dose and the administration of the second dose is 4 hours. In some embodiments of the methods described herein, the dose is administered BID in two divided doses wherein the time interval between the administration of the first dose and the administration of the second dose is 5 hours. In some embodiments of the methods described herein, the dose is administered BID in two divided doses wherein the time interval between the administration of the first dose and the administration of the second dose is 6 hours. In some embodiments of the methods described herein, the dose is administered BID in two divided doses wherein the time interval between the administration of the first dose and the administration of the second dose is 7 hours. In some embodiments of the methods described herein, the dose is administered BID in two divided doses wherein the time interval between the administration of the first dose and the administration of the second dose is 8 hours. In some embodiments of the methods described herein, the dose is administered BID in two divided doses wherein the time interval between the administration of the first dose and the administration of the second dose is 9 hours. In some embodiments of the methods described herein, the dose is administered BID in two divided doses wherein the time interval between the administration of the first dose and the administration of the second dose is 10 hours. In some embodiments of the methods described herein, the dose is administered BID in two divided doses wherein the time interval between the administration of the first dose and the administration of the second dose is 11 hours. In some embodiments of the methods described herein, the dose is administered BID in two divided doses wherein the time interval between the administration of the first dose and the administration of the second dose is 12 hours. In some embodiments of the methods described herein, the dose is administered BID in two divided doses wherein the time interval between the administration of the first dose and the administration of the second dose is 13 hours. In some embodiments of the methods described herein, the dose is administered BID in two divided doses wherein the time interval between the administration of the first dose and the administration of the second dose is 14 hours. In some embodiments of the methods described herein, the dose is administered BID in two divided doses wherein the time interval between the administration of the first dose and the administration of the second dose is 15 hours. In some embodiments of the methods described herein, the dose is administered BID in two divided doses wherein the time interval between the administration of the first dose and the administration of the second dose is 16 hours.

In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, wherein the hematological cancer is myelofibrosis. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is primary myelofibrosis. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, wherein the hematological cancer is primary myelofibrosis. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is post-PV myelofibrosis or Post-ET myelofibrosis. In some aspects the myelofibrosis is post-PV/ET myelofibrosis or myelofibrosis secondary to PV and ET-prognostic model [MYSEC-PM]. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is chronic myeloid leukemia (CML). In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is Chronic neutrophilic leukemia (CNL). In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is blastic plasmacytoid dendritic cell neoplasm (BPDCN). In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is M3 AML. In some embodiments is a method of treating hematological cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the hematological cancer is APL (acute promyelocytic leukemia).

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is relapsed AML, recurrent AML, refractory AML or any combination thereof. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is relapsed AML. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is recurrent AML. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is refractory AML. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is any combination of relapsed AML, recurrent AML, and refractory AML.

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is de novo AML, secondary AML including therapy related AML and AML with myelodysplasia-related changes (AML with MRC), biphenotypic acute Leukemia (also referred to as Acute Leukemia of ambiguous lineage) or AML with recurrent abnormalities. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is de novo AML. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of 450 mg to about 1500 mg, wherein the AML is secondary AML including therapy related AML and AML with myelodysplasia-related changes (AML with MRC). In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is AML with myelodysplasia-related changes (AML with MRC). In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is biphenotypic acute Leukemia (also referred to as acute leukemia of ambiguous lineage). In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is AML with recurrent abnormalities. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is AML with an actionable mutation. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the AML is AML without an actionable mutation.

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is relapsed or refractory MDS. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is classified by the Revised International Prognostic Scoring System (IPSS-R) as low risk MDS. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a administered over a twenty-four hour period dose of about 450 mg to about 1500 mg, wherein the MDS is classified by the Revised International Prognostic Scoring System (IPSS-R) as intermediate risk MDS. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is classified by the Revised International Prognostic Scoring System (IPSS-R) as high risk MDS. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is classified by the Revised International Prognostic Scoring System (IPSS-R) as very high risk MDS. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is selected from the group consisting of MDS with single lineage dysplasia (MDS-SLD), MDS with multilineage dysplasia (MDS-MLD), MDS with ringed sideroblasts (MDS-RS), MDS with ringed sideroblasts with single lineage dysplasia (MDS-RS-SLD), MDS with ringed sideroblasts (MDS-RS), MDS with ringed sideroblasts with multilineage dysplasia (MDS-RS-MLD), MDS with excess blasts 1 and/or 2 (MDS-EB-1, MDS-EB-2), MDS unclassifiable (MDS-U), and MDS with isolated del (5q). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with single lineage dysplasia (MDS-SLD). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with multilineage dysplasia (MDS-MLD). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with Ringed Sideroblasts (MDS-RS). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with Ringed Sideroblasts with single lineage dysplasia (MDS-RS-SLD). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with ringed sideroblasts (MDS-RS). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with Ringed Sideroblasts with multilineage dysplasia (MDS-RS-MLD). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with excess blasts 1 and/or 2 (MDS-EB-1, MDS-EB-2). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS unclassifiable (MDS-U). In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, wherein the MDS is MDS with isolated del (5q).

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the subject is treated irrespective of the subject's mutation or cytogenetic status.

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a complete remission, complete remission without minimal residual disease, complete remission with incomplete hematologic recovery, morphologic leukemia-free state or partial remission, hematological improvement, complete cytogenetic response, transfusion independence, red blood cell transfusion independence or platelet transfusion independence, or eligibility for stem cell transplantation. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a complete remission. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a partial remission. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a hematological improvement. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a complete cytogenetic response. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a transfusion independence. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a red blood cell transfusion independence or platelet transfusion independence. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an eligibility for stem cell transplantation.

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increase in overall survival, an increase in relapse free survival, an increase in event free survival, an increased duration of response or a reduction in cumulative incidence of relapse. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increase in overall survival. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, whereby the subject experiences a therapeutic response, wherein therapeutic response comprises an increase in relapse free survival. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increase in event free survival. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increased duration of response. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a reduction in cumulative incidence of relapse.

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising (a) 4 sequential days of administering the drug to a subject followed by 3 sequential days of no administration, (b) 5 sequential days of administering the drug to a subject followed by 2 sequential days of no administration, (c) a once weekly dosage, or (d) a twice-weekly dosage, and wherein for each of (a)-(d), the dose is administered in a single dose on the day(s) of dosing or administered as two doses, such as separated by 10-12 hours between doses, on the day(s) of dosing. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising 4 sequential days of administering the drug to a subject followed by 3 sequential days of no administration and wherein the dose is administered in a single dose on the day(s) of dosing or administered as two doses, such as separated by 10-12 hours between doses, on the day(s) of dosing. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising 5 sequential days of administering the drug to a subject followed by 2 sequential days of no administration and wherein the dose is administered in a single dose on the days of dosing or administered as two doses, such as separated by 10-12 hours between doses, on the days of dosing. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising a once weekly dosage and wherein the dose is administered in a single dose on the day of dosing or administered as two doses, such as separated by 10-12 hours between doses, on the day of dosing. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising a twice-weekly dosage and wherein the dose is administered in a single dose on the days of dosing or administered as two doses, such as separated by 10-12 hours between doses, on the days of dosing. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein administration regimen is repeated. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a 28 day cycle comprising administration on days 1-4, 8-11, 15-18, and 22-25 of each cycle and wherein the dose is administered in a single dose on the days of dosing or administered as two doses, such as separated by 10-12 hours between doses, on the days of dosing. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a 28 day cycle comprising administration on days 1-4, 8-11, 15-18, and 22-25 of each cycle and the 28 day cycle is repeated at least once, and wherein the dose is administered in a single dose on the days of dosing or administered as two doses, such as separated by 10-12 hours between doses, on the days of dosing. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a 28 day cycle comprising administration on days 1-4, 8-11, 15-18, and 22-25 of each cycle and the 28 day cycle is repeated until relapse, and wherein the dose is administered in a single dose on the days of dosing or administered as two doses, such as separated by 10-12 hours between doses, on the days of dosing.

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered once daily on the days of administration. In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered two times per day on the days of administration.

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein is the pharmaceutical composition administered orally.

In some embodiments is a method of treating myelodysplastic syndrome (MDS) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein is the pharmaceutical composition is administered as one or more tablets or capsules.

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increase in overall survival, an increase in relapse free survival, an increase in event free survival, an increased duration of response or a reduction in cumulative incidence of relapse. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increase in overall survival. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 450 mg to about 1500 mg, whereby the subject experiences a therapeutic response, wherein therapeutic response comprises an increase in relapse free survival. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increase in event free survival. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises an increased duration of response. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein therapeutic response comprises a reduction in cumulative incidence of relapse.

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising (a) 4 sequential days of administering the drug to a subject followed by 3 sequential days of no administration, (b) 5 sequential days of administering the drug to a subject followed by 2 sequential days of no administration, (c) a once weekly dosage, or (d) a twice-weekly dosage, and wherein for each of (a)-(d), the dose is administered in a single dose on the day(s) of dosing or administered as two doses, such as separated by 10-12 hours between doses, on the day(s) of dosing. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising 4 sequential days of administering the drug to a subject followed by 3 sequential days of no administration and wherein the dose is administered in a single dose on the day(s) of dosing or administered as two doses, such as separated by 10-12 hours between doses, on the day(s) of dosing. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising 5 sequential days of administering the drug to a subject followed by 2 sequential days of no administration and wherein the dose is administered in a single dose on the days of dosing or administered as two doses, such as separated by 10-12 hours between doses, on the days of dosing. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising a once weekly dosage and wherein the dose is administered in a single dose on the day of dosing or administered as two doses, such as separated by 10-12 hours between doses, on the day of dosing. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a regimen comprising a twice-weekly dosage and wherein the dose is administered in a single dose on the days of dosing or administered as two doses, such as separated by 10-12 hours between doses, on the days of dosing. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein administration regimen is repeated. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a 28 day cycle comprising administration on days 1-4, 8-11, 15-18, and 22-25 of each cycle and wherein the dose is administered in a single dose on the days of dosing or administered as two doses, such as separated by 10-12 hours between doses, on the days of dosing. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a 28 day cycle comprising administration on days 1-4, 8-11, 15-18, and 22-25 of each cycle and the 28 day cycle is repeated at least once, and wherein the dose is administered in a single dose on the days of dosing or administered as two doses, such as separated by 10-12 hours between doses, on the days of dosing. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered in a 28 day cycle comprising administration on days 1-4, 8-11, 15-18, and 22-25 of each cycle and the 28 day cycle is repeated until relapse, and wherein the dose is administered in a single dose on the days of dosing or administered as two doses, such as separated by 10-12 hours between doses, on the days of dosing.

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 m, whereby the subject experiences a therapeutic response, wherein the pharmaceutical composition is administered once daily on the days of administration. In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein the pharmaceutical composition is administered two times per day on the days of administration.

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein is the pharmaceutical composition administered orally.

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response, wherein is the pharmaceutical composition is administered as one or more tablets or capsules.

In some embodiments is a method of treating acute myeloid leukemia (AML) in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 450 mg to about 1500 mg, whereby the subject experiences a therapeutic response and the subject carries one or more mutations in a locus or multiple loci selected from the group consisting of ABL1, ASXL1, BCOR, BCORL1, BCR, BRAF, CALR, CBFB, CBL, CBLB, CDKN2A, CEBPA, CSF3R, CUX1, DEK, DNMT3A, ETV6, EZH2, FBXW7, FLT3, GATA1, GATA2, GNAS, HRAS, IDH1, IDH2, IKZF1, JAK2, JAK3, KDM6A, KIT, KMT2A, MECOM (EVI1), MLL, MLLT3, MPL, MYD88, MYH11, NOTCH1, NPM1, NUP214, NRAS, PDGFRA, PHF6, PTEN, PTPN11, RAD21, RUNX1, SF3B1, SRSF2, SMC1A, SMC3, STAG2, TET2, TP53, U2AF1, WT1, and ZRSR2. In some embodiments, the subject carries mutations at 2, 3, 4, 5, 6, 7, 8, 9, 10, 11, 12, 13, 14, 15, 16, 17, 18, 19, 20 or more than 20 loci of the group consisting of ABL1, ASXL1, BCOR, BCORL1, BCR, BRAF, CALR, CBFB, CBL, CBLB, CDKN2A, CEBPA, CSF3R, CUX1, DEK, DNMT3A, ETV6, EZH2, FBXW7, FLT3, GATA1, GATA2, GNAS, HRAS, IDH1, IDH2, IKZF1, JAK2, JAK3, KDM6A, KIT, KMT2A, MECOM (EVI1), MLL, MLLT3, MPL, MYD88, MYH11, NOTCH1, NPM1, NUP214, NRAS, PDGFRA, PHF6, PTEN, PTPN11, RAD21, RUNX1, SF3B1, SRSF2, SMC1A, SMC3, STAG2, TET2, TP53, U2AF1, WT1, and ZRSR2.

### Therapeutic Responses

The methods herein include administering a pharmaceutical composition comprising 1-(4-(benzylamino)-5,6,7,8-tetrahydropyrido[2,3-d]pyrimidin-2-yl)-2-methyl-1H-indole-4-carboxamide (Compound 1), or a pharmaceutically acceptable salt thereof, to provide a therapeutic response in to a treated subject. Therapeutic response may depend upon the type of cancer treated as well as the treated subject, stage of the cancer, prior treatments of the subject and other health factors. In some embodiments herein, the cancer treated comprises a hematological cancer, such as AML or MDS, and the therapeutic response is one or more of a complete remission, complete remission without minimal residual disease, complete remission with incomplete hematologic recovery, complete remission with incomplete platelet recovery, composite complete remission, morphologic leukemia-free state or partial remission, hematological improvement, complete cytogenetic response, transfusion independence, red blood cell transfusion independence, platelet transfusion independence, or eligibility for stem cell transplantation. In some embodiments, the therapeutic response also includes or is one or more of an increase in overall survival, an increase in relapse free survival, an increase in event free survival, an increased duration of response or a reduction in cumulative incidence of relapse. In some embodiments, the therapeutic response of a subject treated for multiple myeloma includes or is one or more of sustained MRD-negative, flow MRD-negative, sequencing MRD-negative, imaging plus MRD-negative, stringent complete response, complete response, very good partial response, partial response, minimal response or stable disease, such as by criteria set forth in NCCN Guidelines Version 3.2021 Multiple Myeloma or by criteria set forth in the International Myeloma Working Group response criteria (https://radiopaedia.org/articles/international-myeloma-working-group-response-criteria?lang=us).

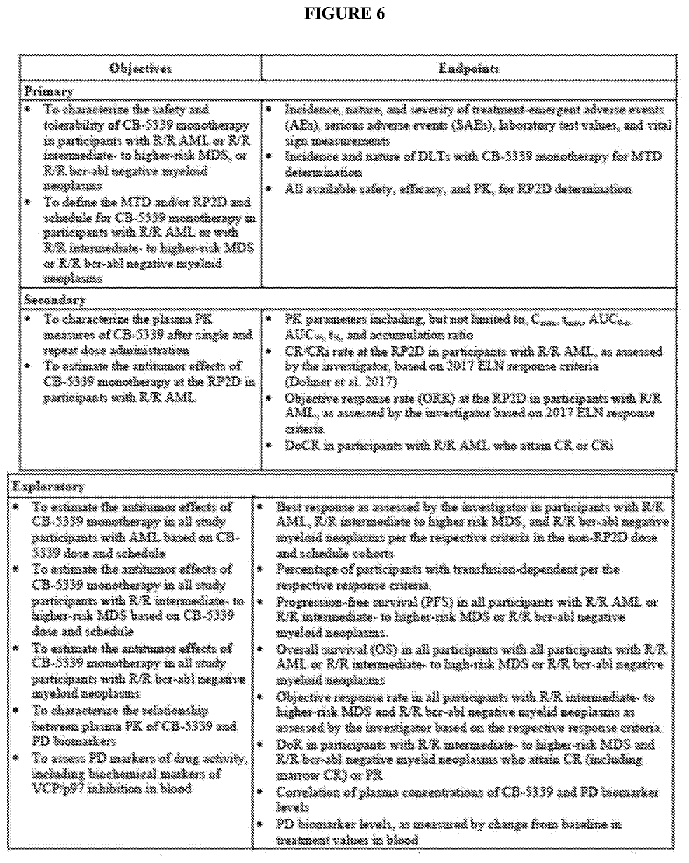

In some embodiments, therapeutic response of a subject to treatment with a pharmaceutical composition comprising 1-(4-(benzylamino)-5,6,7,8-tetrahydropyrido[2,3-d]pyrimidin-2-yl)-2-methyl-1H-indole-4-carboxamide (Compound 1), or a pharmaceutically acceptable salt thereof is performed based on physical examination. In some embodiments, therapeutic response of a subject to treatment with a pharmaceutical composition comprising 1-(4-(benzylamino)-5,6,7,8-tetrahydropyrido[2,3-d]pyrimidin-2-yl)-2-methyl-1H-indole-4-carboxamide (Compound 1), or a pharmaceutically acceptable salt thereof is performed based on peripheral blood and bone marrow aspirate/biopsy using ELN (Dohner H, Estey E, Grimwade D, et al. Diagnosis and management of AML in adults: 2017 ELN recommendations from an international expert panel. Blood. 2017; 129(4):424-47) for AML, IWG criteria (Cheson B D, Greenberg P L, Bennett J M, et al. Clinical application and proposal for modification of the International Working Group (IWG) response criteria in myelodysplasia. Blood. 2006; 108(2):419-25) for MDS, IWG-MRT and ELN Response Criteria for PV and ET (Barosi G, Mesa R, et al. Revised response criteria for polycythemia vera and essential thrombocythemia; and ELN and IWG-MRT consensus project. Blood. 2013; 121(23): 4778-4781), IWG-MRT and ELN Response Criteria for MF (Tefferi A, Cervantes F, Mesa R, et al. Revised response criteria for myelofibrosis: International Working Group-Myeloproliferative Neoplasm Research and Treatment (IWG-MRT) and European LeukemiaNet (ELN) consensus report. Blood. 2013; 122 (8): 1395-1398.), or MDS/MPN response criteria (Savona M R, Malcovati L, Komrokji R, et al. An international consortium proposal of uniform response criteria for myelodysplastic/myeloproliferative neoplasms (MDS/MPN) in adults. Blood. 2015; 125(12): 1857-1865) for AML, MDS, bcr-abl negative MPN, or MDS/MPN respectively. In some embodiments, therapeutic response of a subject to treatment with a pharmaceutical composition comprising 1-(4-(benzylamino)-5,6,7,8-tetrahydropyrido[2,3-d]pyrimidin-2-yl)-2-methyl-1H-indole-4-carboxamide (Compound 1), or a pharmaceutically acceptable salt thereof is assessed based on one or more criteria described in FIG. 6.

### Combination Therapies

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, wherein the treatment further includes administration of a second therapeutic agent, and whereby the subject experiences a therapeutic response with the combination that is greater than the response with either agent alone.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, wherein the treatment further includes administration of a second therapeutic agent, and whereby the subject experiences a therapeutic response with the combination that is greater than the response with either agent alone. In some embodiments, a therapeutic response with the combination is additive of the response with either agent alone. In some embodiments, a therapeutic response with the combination is greater than additive of the response with either agent alone. In some embodiments, a therapeutic response with the combination is synergistic as compared to the response with either agent alone. Syngerism and additive effects may be assessed by models available in the art. Exemplary models for assessing synergy and additive effects include the Bliss independence model and Loewe additivity model (see Greco, W., Unkelbach, H. D., Poch, G., Suhnel, J., Kundi, M., & Bodeker, W. (1992). Consensus on concepts and terminology for combined-action assessment: the Saariselka agreement. Arch Complex Environ Stud, 4(3), 65-9).

Exemplary second therapeutic agents for use in combination with 1-(4-(benzylamino)-5,6,7,8-tetrahydropyrido[2,3-d]pyrimidin-2-yl)-2-methyl-1H-indole-4-carboxamide include one or more of a chemotherapy agent such as nucleic acid analog and a nucleic acid synthesis inhibitor, a tyrosine kinase inhibitor, a DNA damage repair inhibitor, an inhibitor of ATM, an inhibitor of ATR, an inhibitor of PARP, an inhibitor of Chk1, a proteasome inhibitor, an inhibitor of FLT3, an inhibitor of IDH1, an inhibitor of IDH2, an immune oncology agent or an immune modulation agent, a p53 targeted agent, a BCl-2 inhibitor, a BH3 mimic, a DNA-PK inhibitor, and a NEDD-8, inhibitor.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a DNA damaging agent, a hypomethylating agent, an agent that interferes with DNA synthesis or an agent that interferes with DNA replication. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a DNA damaging agent. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a hypomethylating agent. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an agent that interferes with DNA synthesis. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an agent that interferes with DNA replication.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 25 mg to about 2000 mg or of about 450 mg to about 1500 mg, whereby the subject experiences a therapeutic response, wherein the treatment further includes administration of a second therapeutic agent and the second therapeutic agent is decitabine, azacytidine, or cytarabine. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose of about 25 mg to about 2000 mg or of about 450 mg to about 1500 mg, whereby the subject experiences a therapeutic response, wherein the treatment further includes administration of a second therapeutic agent and the second therapeutic agent is decitabine. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is azacytidine. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is cytarabine.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is cytarabine dosed in a 7+3 regimen comprising 7 days of cytarabine and 3 days of an anthracycline antibiotic selected from daunorubicin, doxorubicin, idarubicin, and mitoxantrone. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is cytarabine dosed in a 7+3 regimen comprising 7 days of cytarabine and 3 days of an anthracycline antibiotic. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is cytarabine dosed in a 7+3 regimen comprising 7 days of cytarabine and 3 days of daunorubicin. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is cytarabine dosed in a 7+3 regimen comprising 7 days of cytarabine and 3 days of doxorubicin. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is cytarabine dosed in a 7+3 regimen comprising 7 days of cytarabine and 3 days of idarubicin. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is cytarabine dosed in a 7+3 regimen comprising 7 days of cytarabine and 3 days of mitoxantrone.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a tyrosine kinase inhibitor.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a DNA damage repair inhibitor.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of ATM, ATR, PARP, or Chk1. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of ATM, such as AZD6738. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of ATR, such as AZDO156.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of poly ADP ribose polymerase (PARP). In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of PARP selected from the group consisting of talazoparib, niraparib, and olaparib. In some embodiments of the method, the PARP inhibitor comprises talazoparib or an analog thereof. In some embodiments of the method, the PARP inhibitor comprises niraparib or an analog thereof. In some embodiments of the method, the PARP inhibitor comprises olaparib or an analog thereof.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of Chk1.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a proteasome inhibitor. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is Velcade (bortezomib). In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is Kyprolis (carfilzomib). In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is Ninlaro (ixazomib). In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of NEDD-8. In some embodiments, the inhibitor of NEDD-8 is pevonedistat.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is lenalidomide, dexamethasone, pomalidomide, or a combination thereof, such as where one of the agents in the combination is dexamethasone. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is lenalidomide. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is pomalidomide. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is dexamethasone. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a combination of one or more of lenalidomide, pomalidomide and dexamethasone.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is selected from a FLT3 inhibitor, IDH1 inhibitor, IDH2 inhibitor, hedgehog pathway inhibitor, an anti-CD33 antibody, a purine analog, and a Bcl-2 inhibitor. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of FLT3. In some embodiments of the method, the FLT3 inhibitor comprises gilteritinib, midostaurin, or analogs thereof. In some embodiments of the method, the FLT3 inhibitor comprises gilteritinib or an analog thereof. In some embodiments of the method, the FLT3 inhibitor comprises midostaurin or analog thereof. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of IDH1. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of IDH2. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of hedgehog pathway inhibitor. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of hedgehog pathway inhibitor, wherein the hedgehog pathway inhibitor is glasdegib. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an anti-CD33 antibody. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an anti-CD33 antibody, wherein the anti-CD33 antibody is gemtuzumab ozogamicin. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is Sarclisa (isatuximab) or a CD38 directed monoclonal antibody. In some embodiments, the anti-CD38 antibody is Sarclisa (isatuximab). In some embodiments, the anti-CD38 antibody is daratumumab.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a menin inhibitor. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is the menin inhibitor SNDX-5613. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is the menin inhibitor KO-539. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a selective inhibitor of nuclear export. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is the selective inhibitor of nuclear export Selinexor.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a purine analog. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a purine analog, wherein the purine analog is fludarabine. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an inhibitor of Bcl-2. In some embodiments of the method, the Bcl-2 inhibitor is a BH3-mimetic. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a Bcl-2 inhibitor, wherein the Bcl-2 inhibitor is venetoclax. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is a DNA protein kinase inhibitor. In some such embodiments, the DNA protein kinase inhibitor is nedisertib. In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent acts on p53 or a mutant form of p53. In some such embodiments, the second therapeutic agent is APR-246.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an immune oncology agent.

In some embodiments is a method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose administered over a twenty-four hour period of about 25 mg to about 2000 mg or of about 450 mg to about 1500 mg, whereby the subject experiences a therapeutic response, wherein the treatment further includes administration of a second therapeutic agent and the second therapeutic agent is an immune modulation agent.

### Dosage

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered over a 24-hour period to a subject in need thereof, whereby the subject experiences a therapeutic response, is about 450 mg, about 455 mg, about 460 mg, about 465 mg, about 470 mg, about 475 mg, about 480 mg, about 485 mg, about 490 mg, about 495 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, or about 1500 mg, including increments therein.

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered to a subject in need thereof, whereby the subject experiences a therapeutic response, is administered in unit dosage, such as in a tablet or capsule, wherein the unit dosage of 1-(4-(benzylamino)-5,6,7,8-tetrahydropyrido[2,3-d]pyrimidin-2-yl)-2-methyl-1H-indole-4-carboxamide in each tablet or capsule is or is about 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg or 1000 mg.

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered over a 24-hour period to a subject in need thereof, whereby the subject experiences a therapeutic response, is from about 450 mg to about 1500 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 450 mg to about 1000 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 450 mg to about 900 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 450 mg to about 750 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 450 mg to about 600 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 450 mg to about 525 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 600 mg to about 1000 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 600 mg to about 900 mg administered in one or two administrations. In some embodiments of the methods disclosed herein, the dose of Compound 1, or a pharmaceutically acceptable salt thereof, administered to a subject in need thereof, whereby the subject experiences a therapeutic response, is from about 600 mg to about 750 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 600 mg to about 700 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 900 mg to about 1500 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 900 mg to about 1250 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 900 mg to about 1000 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 1000 mg to about 1500 mg administered in one or two administrations. In some embodiments of the methods disclosed herein, the dose of Compound 1, or a pharmaceutically acceptable salt thereof, administered over a 24-hour period to a subject in need thereof, whereby the subject experiences a therapeutic response, is from about 1000 mg to about 1250 mg administered in one or two administrations.

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered to a subject in need thereof, whereby the subject experiences a therapeutic response, is administered orally. In some embodiments of the methods disclosed herein, the dose of Compound 1, or a pharmaceutically acceptable salt thereof, administered to a subject in need thereof, whereby the subject experiences a therapeutic response, is administered with food. In some embodiments of the methods disclosed herein, the dose of Compound 1, or a pharmaceutically acceptable salt thereof, administered to a subject in need thereof, whereby the subject experiences a therapeutic response, is administered without food.

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered to a subject in need thereof, whereby the subject experiences a therapeutic response, is administered once per day. In some embodiments of the methods disclosed herein, the dose of Compound 1, or a pharmaceutically acceptable salt thereof, administered to a subject in need thereof, whereby the subject experiences a therapeutic response, is administered twice per day. In some embodiments of the methods disclosed herein, the dose of Compound 1, or a pharmaceutically acceptable salt thereof, administered to a subject in need thereof, whereby the subject experiences a therapeutic response, is administered three times per day. In some embodiments of the methods disclosed herein, the dose of Compound 1, or a pharmaceutically acceptable salt thereof, administered to a subject in need thereof, whereby the subject experiences a therapeutic response, is administered four times per day.

In some embodiments, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered to a subject in need thereof, whereby the subject experiences a therapeutic response, is administered with a therapeutic “holiday”, that is a period of days in between continuous doses. For example, the dose of Compound 1 is dosed for 1-7 days (the days “on”) and then a period of 1-7 days is the “holiday” where no dosage of Compound 1 is administered to the subject (the days “off”). In some embodiments, the dose of Compound 1, or a pharmaceutically acceptable salt thereof, administered to a subject in need thereof, whereby the subject experiences a therapeutic response, is administered as 4 days on/3 days off, 5 days on/3 days off, 5 days on/2 days off, or 4 days on/2 days off. In some embodiments, the cycle of on/off dosage is repeated, such that the dosage of Compound 1 is administered in 2, 3, 4, 5 or more than 5 cycles of on/off schedule.

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered to a subject in need thereof, whereby the subject experiences a therapeutic response, is administered once every two days. In some embodiments of the methods disclosed herein, the dose of Compound 1, or a pharmaceutically acceptable salt thereof, administered to a subject in need thereof, whereby the subject experiences a therapeutic response, is administered once every three days. In some embodiments of the methods disclosed herein, the dose of Compound 1, or a pharmaceutically acceptable salt thereof, administered to a subject in need thereof, whereby the subject experiences a therapeutic response, is administered once every four days. In some embodiments of the methods disclosed herein, the dose of Compound 1, or a pharmaceutically acceptable salt thereof, administered to a subject in need thereof, whereby the subject experiences a therapeutic response, is administered once every five days. In some embodiments of the methods disclosed herein, the dose of Compound 1, or a pharmaceutically acceptable salt thereof, administered to a subject in need thereof, whereby the subject experiences a therapeutic response, is administered once every six days. In some embodiments of the methods disclosed herein, the dose of Compound 1, or a pharmaceutically acceptable salt thereof, administered to a subject in need thereof, whereby the subject experiences a therapeutic response, is administered once every week.

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered to a subject in need thereof, whereby the subject experiences a therapeutic response, is administered for four days on and three days off.

In some embodiments of the methods disclosed herein, the dose of Compound 1 (1-(4-(benzylamino)-5,6,7,8-tetrahydropyrido[2,3-d]pyrimidin-2-yl)-2-methyl-1H-indole-4-carboxamide), or a pharmaceutically acceptable salt thereof, administered to a subject in need thereof, where the subject has undergone at least one prior therapy.

### Pharmaceutical Compositions

In some embodiments, the 1-(4-(benzylamino)-5,6,7,8-tetrahydropyrido[2,3-d]pyrimidin-2-yl)-2-methyl-1H-indole-4-carboxamide pharmaceutical compositions described herein comprise 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, and at least one pharmaceutically acceptable excipient. In some embodiments, the pharmaceutical compositions described herein comprise 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, and at least one pharmaceutically acceptable excipient, in a solid dosage form. In some embodiments, the pharmaceutical compositions described herein comprise crystalline 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, and at least one pharmaceutically acceptable excipient, in a solid dosage form. In some embodiments, the pharmaceutical compositions described herein comprise 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, and at least one pharmaceutically acceptable excipient, in a solid dosage form, wherein the solid dosage form is selected from a powder, a tablet, a bite-disintegration tablet, a chewable tablet, a caplet, a capsule, a gelcap, an effervescent powder, a rapid-disintegration tablet, an abuse-deterrent tablet, a modified release tablet, a modified release caplet, a modified release capsule, and an aqueous suspension produced from a powder. In some embodiments, the pharmaceutical compositions described herein comprise 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, and at least one pharmaceutically acceptable excipient, in a solid dosage form, wherein the solid dosage form is a capsule. In some embodiments, the pharmaceutical compositions described herein comprise 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, and at least one pharmaceutically acceptable excipient, in a solid dosage form, wherein the solid dosage form is a tablet.

### Excipients

Suitable optional excipients for use in the 1-(4-(benzylamino)-5,6,7,8-tetrahydropyrido[2,3-d]pyrimidin-2-yl)-2-methyl-1H-indole-4-carboxamide pharmaceutical compositions described herein include any commonly used excipients in pharmaceutics and are selected on the basis of compatibility with the active pharmaceutical agent and the release profile properties of the desired dosage form. Excipients include, but are not limited to, binders, fillers, flow aids, disintegrants, lubricants, glidants, polymeric carriers, plasticizers, stabilizers, surfactants, and the like. A summary of excipients described herein, may be found, for example in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), herein incorporated by reference in their entirety.

Fillers or diluents increase bulk in the pharmaceutical composition. Such compounds include e.g., lactose; starch; mannitol; sorbitol; dextrose; microcrystalline cellulose such as Avicel®; silicified microcrystalline cellulose such as ProSolv® HD90; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); hydroxypropylmethylcellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; sodium chloride; inositol; bentonite; and the like. In some embodiments, the pharmaceutical compositions described herein comprise two fillers. In some embodiments of the pharmaceutical compositions described herein, the first filler and second filler are selected from lactose, mannitol, dicalcium phosphate, microcrystalline cellulose, silicified microcrystalline cellulose, starch, and pregelatinized starch (Starch 1500). In some embodiments of the pharmaceutical compositions described herein, the first filler and second filler are independently selected from lactose, mannitol, microcrystalline cellulose, and silicified microcrystalline cellulose.

Binders impart cohesiveness to solid oral dosage form compositions: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Agoate HS-LF and HS), hydroxyethylcellulose, hydroxypropylcellulose (e.g., Klucel®), ethylcellulose (e.g., Ethocel®), microcrystalline cellulose (e.g., Avicel®), silicified microcrystalline cellulose such as ProSolv® HD90, microcrystalline dextrose, amylose, magnesium aluminum silicate, polysaccharide acids, bentonites, gelatin, polyvinyl pyrrolidone/vinyl acetate copolymer, crospovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinyl pyrrolidone (e.g., Povidone® CL, Kollidon® CL, Polyplasdone® XL-10, and Povidone® K-12), larch arabogalactan, Veegum®, polyethylene glycol, waxes, sodium alginate, and the like. In some embodiments, the binder is hypromellose, hydroxypropyl cellulose, or ethyl cellulose.

Glidants improve the flow characteristics of a powder mixtures. Such compounds include, e.g., colloidal silicon dioxide such as Cab-o-Sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®) and the like. In some embodiments of the pharmaceutical compositions described herein, the glidant is colloidal silicon dioxide or talc. In some embodiments, the glidant is talc. In some embodiments, the glidant is colloidal silicon dioxide.

Lubricants are compounds which prevent, reduce, or inhibit adhesion or friction of materials. Exemplary lubricants include, e.g., stearic acid; calcium hydroxide, talc; paraffin; a hydrocarbon such as mineral oil, or hydrogenated vegetable oil such as hydrogenated soybean oil (Sterotex®), Lubritab®, Cutina®; higher fatty acids and their alkali-metal and alkaline earth metal salts, such as aluminum, calcium, magnesium, zinc, stearic acid, sodium stearate, calcium stearate, magnesium stearate, glycerol, talc, waxes, Stearowet®, boric acid, sodium acetate, leucine, a polyethylene glycol or a methoxypolyethylene glycol such as Carbowax™, sodium oleate, glyceryl behenate (Compitrol 888®), glyceryl palmitostearate (Precirol®), colloidal silica such as Syloid™ Carb-O-Sil®, a starch such as corn starch, silicone oil, a surfactant, and the like. Hydrophilic lubricants include, e.g., sodium stearyl fumarate (currently marketed under the trade name PRUV®), polyethylene glycol (PEG), magnesium lauryl sulfate, sodium lauryl sulfate (SLS), sodium benzoate, sodium chloride, and the like. In some embodiments of the pharmaceutical compositions described herein, the lubricant is magnesium stearate, stearic acid, or sodium stearyl fumarate. In some embodiments, the lubricant is stearic acid. In some embodiments, the lubricant is sodium stearyl fumarate. In some embodiments, the lubricant is magnesium stearate.

Disintegrants facilitate breakup or disintegration of the pharmaceutical formulation after administration. Examples of disintegrants include a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinyl pyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a resin such as a cation-exchange resin; citrus pulp; sodium lauryl sulfate; sodium lauryl sulfate in combination starch; and the like. In some embodiments of the pharmaceutical compositions described herein, the disintegrant is selected from povidone, crospovidone, hypromellose, croscarmellose sodium, hydroxypropyl cellulose, and polyvinyl alcohol. In some embodiments, the disintegrant is croscarmellose sodium. In some embodiments, the disintegrant is polyvinyl alcohol. In some embodiments, the disintegrant is hydroxypropyl cellulose. In some embodiments, the disintegrant is hypromellose. In some embodiments, the disintegrant is povidone. In some embodiments, the disintegrant is crospovidone.

In some embodiments, the pharmaceutical compositions described herein include one or more pH-adjusting agents or buffering agents. In some embodiments, the pharmaceutical formulation comprises a buffer selected from acetates, carbonates, phosphates, citrates, and glutamates. In some embodiments, the buffer is selected from potassium dihydrogen phosphate, sodium bicarbonate, magnesium carbonate, sodium citrate, sodium dihydrogen phosphate, dipotassium monohydrogen phosphate, and disodium monohydrogen phosphate. In some embodiments, buffers are included in an amount required to maintain pH of the pharmaceutical formulation in an acceptable range.

In some embodiments, a film coating is provided around the pharmaceutical composition. In some embodiments, the coating of 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, is an immediate release coating. In some embodiments, the immediate release coating comprises hydroxypropyl methyl cellulose (HPMC), with or without plasticizer, and with or without surfactants and anti-foaming agents (clear or pigmented or dyed). In some embodiments, the coating of 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, is an immediate release coating with a moisture barrier. In some embodiments, the film coating is Opadry AMB II Beige. In some embodiments, the immediate release coating with a moisture barrier comprises polyvinyl alcohol (PVA), with or without plasticizer, with or without surfactants and anti-foaming agents (clear or pigmented or dyed). In some embodiments, the compositions are formulated into particles (for example for administration by capsule) and some or all of the particles are coated. In some embodiments, the compositions are formulated into particles (for example, for administration by capsule) and some or all of the particles are microencapsulated. In some embodiments, the compositions are formulated into particles (for example, for administration by capsule) and some or all of the particles are not microencapsulated and are uncoated.

In some embodiments, the compositions described herein are delivered using a pulsatile dosage form. A pulsatile dosage form is capable of providing one or more immediate release pulses at predetermined time points after a controlled lag time or at specific sites. Many other types of controlled release systems known to those of ordinary skill in the art and are suitable for use with the compositions described herein. Examples of such delivery systems include, e.g., polymer-based systems, such as polylactic and polyglycolic acid, polyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014, and 6,932,983, each of which is specifically incorporated by reference.

Stabilizers include compounds such as any anti-oxidation agents, e.g., butylated hydroxytoluene (BHT), sodium ascorbate, and tocopherol; buffers, acids, and the like.

Surfactants include compounds such polysorbates, poloxamers, bile salts, glyceryl monostearate, sodium lauryl sulfate, sorbitan monooleate, polyoxyethylene sorbitan monooleate, copolymers of ethylene oxide and propylene oxide, and d-α-tocopheryl polyethylene glycol succinate (Vitamin E TPGS). In some embodiments, the surfactant is selected from Soluplus, PEG4000, PEG6000, Poloxamer 6200, and Kolliphor P407 micro. In some embodiments, the surfactant is a poloxamer. In some embodiments, the surfactant is Kolliphor P407 micro.

The aforementioned excipients are given as examples only and are not meant to include all possible choices. Other suitable excipient classes include coloring agents, granulating agents, preservatives, anti-foaming agents, plasticizers, and the like. Additionally, many excipients can have more than one role or function, or can be classified in more than one group; the classifications are descriptive only, and are not intended to limit any use of a particular excipient.

### Kits/Articles of Manufacture

For use in the methods described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Packaging materials for use in packaging pharmaceutical products include, e.g., U.S. Pat. No. 5,323,907. Examples of pharmaceutical packaging materials include, but are not limited to, blister packs, bottles, tubes, bags, containers, bottles, and any packaging material suitable for a selected formulation and intended mode of administration and treatment.

In some embodiments, the pharmaceutical compositions of Compound 1 described herein, are presented in a package or dispenser device which may contain one or more unit dosage forms containing the active ingredient. The pharmaceutical composition of Compound 1 described herein is packaged alone, or packaged with another compound or another ingredient or additive. In some embodiments, the package contains one or more containers filled with one or more of the ingredients of the pharmaceutical composition. In some embodiments, the package comprises metal or plastic foil, such as a blister pack. In some embodiments, the package or dispenser is accompanied with a notice associated with the container in form prescribed by a governmental agency regulating the manufacture, use, or sale of pharmaceuticals, which notice is reflective of approval by the agency of the form of the drug for human or veterinary administration. In some embodiments, such notice, for example, is the labeling approved by the U.S. Food and Drug Administration for prescription drugs, or the approved product insert.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### EXAMPLES

### List of Abbreviations

As used throughout the description of the invention, the following abbreviations, unless otherwise indicated, shall be understood to have the following meanings: AML=acute myeloid leukemia; AUC_{∞}=area under the plasma concentration-time curve from time zero to 24 hours; AUC₀₋ₜ=area under the concentration-time curve from time zero to the last quantifiable timepoint; Cₘₐₓ=maximum observed plasma concentration; CR=complete remission; CRi=complete remission with incomplete hematologic recovery; DoCR=duration of complete remission/complete remission with incomplete hematologic recovery; DoR=duration of remission; ELN=European LeukemiaNet; IWG=International Working Group; MTD=maximum tolerated dose; MDS=myelodysplastic syndrome; PD=pharmacodynamic; PK=pharmacokinetic; PR=partial remission; RP2D=recommended Phase 2 dose; RR=relapsed/refractory; t½=half-life; tₘₐₓ=time to maximum concentration.

### Example 1: Preparation of Compound 1

The preparation of Compound 1 is disclosed in U.S. Pat. No. 9,828,363, the content of which is incorporated by reference in its entirety.

### Example 2: Phase I Study to Evaluate the Safety and Pharmacokinetic Profiles of Compound 1 in Participants with Relapsed/Refractory Acute Myeloid Leukemia or Relapsed/Refractory Intermediate or High-Risk Myelodysplastic Syndrome

A first-in-human (FIH) study was designed and performed to evaluate the safety, pharmacokinetics (PK), preliminary efficacy and pharmacodynamic (PD) profiles of escalating doses of Compound 1 monotherapy in adult participants with R/R AML or R/R intermediate- to high-risk MDS and to identify the recommended Phase 2 dose (RP2D) and dosing schedule.

### Study Design

The study was designed as a multicenter, open-label Phase 1 study of orally administered Compound 1 in participants with R/R AML or participants with R/R intermediate- to high-risk MDS. The study includes two parts: 1) a Dose Escalation phase in participants with R/R AML, or R/R intermediate- to high-risk MDS and 2) a Dose Expansion phase in participants with R/R AML for whom there is no standard of care therapy available that is likely to lead to disease remission. Additional cohorts for participants with R/R intermediate- to high-risk MDS following hypomethylating agents or other AML cohorts may be added at a later time.

During the first part, Compound 1 was administered orally QD on the 4/3 schedule in successive 28-day cycles until progressive disease or intolerable toxicity ensues. Similar dosing may be used in the dose expansion part of the study. BID dosing may be also tested depending on initial PK assessments. Participants were evaluated regularly for safety assessments including physical examinations and laboratory testing. Bone marrow aspirates and/or biopsies are performed at regular intervals, including for assessment of tumor response as per 2017 European LeukemiaNet (ELN) response criteria for AML or 2006 revised International Working Group (IWG) response criteria for MDS. Peripheral blood samples are collected in Cycle 1 for intensive PK testing and PD biomarker assessments. Additional intensive PK testing is collected for any participants undergoing intra-patient dose escalation.

### Dose Escalation Phase

The study was commenced with the Dose Escalation Phase using an accelerated titration design to define the maximum tolerated dose (MTD) and/or RP2D of Compound 1 monotherapy in participants with R/R AML or R/R intermediate- to high-risk MDS. During this portion of the study, consented eligible participants were enrolled into sequential cohorts of increasing doses of Compound 1. The starting dose of Compound 1 was 25 mg orally administered QD using a 4/3 weekly dosing schedule. A cycle consists of 28 days and the first cycle comprise the DLT observation period. Table 1 shows the dosing cohorts.

TABLE 1

| Dose Level | Dose Cohort (Once-Daily [QD] Dosing) | |
|---|---|---|
| 1 | 25 | mg |
| 2 | 50 | mg |
| 3 | 100 | mg |
| 4 | 175 | mg |
| 5 | 275 | mg |
| 6 | 350 | mg |
| 7 | 450 | mg |
| 8 | 600 | mg |
| 9 | 750 | mg |
| 10 | 900 | mg |

Single-participant cohorts were used for initial dose escalation during the accelerated phase. When the first instance of a ≥Grade 2 non-hematologic adverse event is observed (during the first cycle) at any dose level, the accelerated phase will end, and dose escalation will be changed to a 3+3 design. In all of the above circumstances, the reference to non-hematologic AEs includes all AEs considered possibly related to study intervention.

When the 3+3 design is utilized, a minimum of 3 participants will be enrolled into a dose cohort (unless 2 DLTs occur within the first 2 participants). If no DLTs are observed after the last participant in the cohort completes the 28-day DLT observation period (i.e., Cycle 1), the study will proceed with dose escalation to the next cohort following safety review. If 1 of 3 participants experiences a DLT during the first cycle, 3 additional participants will be enrolled in that cohort. If none of the additional 3 participants experience a DLT, dose escalation may continue to the next cohort following safety review. If 2 or more participants in a cohort experience DLTs during the first cycle, dose escalation will be halted and the next lower dose level will be declared the MTD. Alternatively, a dose level intermediate between the dose level exceeding MTD and the previous dose level may be explored and declared the MTD if <2 out of 6 participants experience a DLT at that dose. If the MTD cohort included only 3 participants, an additional 3 participants will be enrolled at that dose level to confirm that <2 of 6 participants experience a DLT at that dose.

If there are additional participants in the screening process at the time the last participant in the dose cohort begins treatment, an additional participant may be enrolled into the cohort as long as the participant can begin treatment within 1 week of the last enrolled participant and have received approval from the Medical Monitor.

After each cohort has completed the DLT window, the safety data and any available PK data will be evaluated to determine whether dose escalation can occur, additional participants should be enrolled in a dose cohort, an intermediate dose should be assessed, or BID dosing should be tested (Table 2).

At the conclusion of the 900 mg QD cohort, subjects were enrolled in cohorts for BID dosing, starting with the 300 mg BID dosing.

TABLE 2

| Dose Level | Dose Cohort (Twice-Daily [BID] Dosing) |
|---|---|
| 1 | 225 mg BID |
| 2 | 300 mg BID |
| 3 | 350 mg BID |
| 4 | 450 mg BID |

To optimize the number of participants treated at a potentially clinically relevant dose, intra-participant dose escalation will be permitted after Cycle 1 provided that the participant tolerated the current dose and with approval of the Medical Monitor. Dose escalation will be permitted to any previously cleared dose level. There are no limits on the number of dose escalations that can occur for a participant.

An alternative dosing schedule (e.g., once a week or twice a week dosing) may also be assessed after the MTD and/or RP2D for 4/3 dosing schedule has been defined. Evaluation of the alternative schedule may be initiated earlier if indicated by pharmacokinetic profiles or if clinically significant toxicities (e.g., significant number of Grade 2 AEs or study treatment holds/withdrawals due to intolerability) occur with the 4/3 dosing schedule prior to defining the MTD or RP2D with a 4/3 dosing schedule. The alternative dosing schedule will be defined based on the cumulative safety and PK data available and will be outlined in a formal amendment to the protocol.

The study procedures for these additional participant(s)/cohort(s) will be the same as that described for other study participants/cohorts.

### Selection of the RP2D

At the completion of the dose escalation phase of the study, the following data will be available in determining a RP2D for the expansion phase of the study:
MTD, which is defined as the dose in which the rate of the DLTs is ≤17% in at least 6 participants during the 1^{st} cycle
Delayed DLTs (DLTs occurring beyond the 1^{st} cycle)
Predominate Dose Limiting Toxicity
Overall safety profile
Pharmacokinetics from each cohort
Number of participants requiring discontinuation or dose reductions of study drug

### Expansion Phase

Once the RP2D has been identified, a cohort of approximately 38 additional participants who have R/R AML will be treated at the RP2D (and schedule) to further confirm the safety and to assess the preliminary activity in participants. Depending on emergent data from within this study or from other nonclinical or external data, the Sponsor may elect to add separate cohorts for participants with R/R intermediate- to high-risk MDS or specific subtypes of AML, which may increase the sample size of the Expansion Phase.

After a total of 20 participants with R/R AML are enrolled into a cohort at the RP2D and schedule and treated for at least 2 cycles, the Sponsor will assess the preliminary efficacy and safety data and the benefit/risk profile to determine whether to stop the study for development of Compound 1 as monotherapy in AML. If, despite meeting a futility threshold for Compound 1 monotherapy, an ORR or other parameter suggestive of activity is observed, dose escalation and dose expansion cohorts in combination with an approved therapy may be included by a formal protocol amendment.

### Duration of Study Intervention

Compound 1 will be administered orally QD on the weekly 4/3 schedule for 1 cycle of 28 days. Participants will be evaluated for safety and tolerability and their tumor status will be evaluated after completion of Cycle 1. Participants who do not incur unacceptable toxicity from the study therapy will be permitted to receive a second cycle of study therapy, after which they will be reevaluated for safety, tolerability and efficacy. Participants without unacceptable toxicity at the end of Cycle 2, and who are determined by the investigator to be deriving benefit from study therapy (e.g., have no evidence of disease progression for MDS participants or at least PR for participants with AML) will be permitted to remain on-study therapy until disease progression, unacceptable toxicity, completion of treatment, or other criteria for withdrawal are met, whichever occurs first. Participants will return to the clinical site for an End of Treatment (EoT) visit 30 days (±5 days) after their last 28 day treatment cycle.

After termination of treatment, participants will continue on study for long term survival follow-up every 3 months (±2 weeks), unless they withdraw consent for further follow-up.

### Participants

Approximately 50-60 total participants will be enrolled and treated in the Dose Escalation phase and Dose Expansion phase, including approximately 38 R/R AML participants planned to be enrolled and treated in the Dose Expansion phase.

### Dosing

Compound 1 will be administered orally QD on the 4/3 dosing schedule (4 days on, 3 days off) in successive 28-day cycles until progressive disease or intolerable toxicity ensues. BID dosing may be also tested depending on initial PK assessments.

### Criteria:

### Inclusion Criteria:

Male or female and ≥18 years of age at the time of signing the consent form.
One of the following advanced hematologic malignancies including:
Dose Escalation and Expansion:
Relapsed or refractory AML as defined by 2016 WHO criteria (Arber et al. 2016) and are not candidates for curative therapies such as allogeneic hematopoietic cell transplant or for whom there is no standard of care therapy available that is likely to lead to disease remission according the investigator.
Dose Escalation Only:
MDS high-very high risk by the revised international scoring system for evaluating prognosis in myelodysplastic syndromes that is recurrent or refractory or the participant is intolerant to established therapy known to provide clinical benefit for their condition (e.g., relapsed following treatment with hypomethylating agent or lack of response after ≥4 cycles), according to treating physician. Potential participants which meet the criteria for intermediate risk may be considered with approval by the medical Monitor if the participant has severe cytopenia(s) and/or elevated bone marrow blast counts.
Adequate organ function defined as:
Serum creatinine ≤1.5 mg/dL or an estimated glomerular filtration rate of ≥60 mL/min as calculated by the Cockcroft-Gault glomerular filtration rate equation
Total bilirubin ≤1.5×the upper limit of normal (ULN) unless considered due to Gilbert's disease or leukemic disease.
Aspartate aminotransferase (AST)≤3×the ULN; alanine aminotransferase (ALT)≤3×the ULN. Levels of AST and/or ALT ≤5×the ULN may be acceptable for participants with known leukemic involvement of the liver after discussion with the study medical monitor.
Eastern Cooperative Oncology Group (ECOG) performance status ≤2.
Contraceptive use by men or women should be consistent with local regulations regarding the methods of contraception for those participating in clinical studies. If of childbearing potential, agree to use an effective barrier method of birth control (i.e., latex condom, diaphragm, cervical cap, etc.) to avoid pregnancy during the study and 90 days after the last dose of Compound 1. Female participants of childbearing potential need a negative serum or urine pregnancy test within 7 days of study enrollment. Non-childbearing is defined as ≥1 year postmenopausal or surgically sterilized.
Capable of giving signed informed consent as described in Appendix 1 which includes compliance with the requirements and restrictions listed in the informed consent form (ICF) and in this protocol.

### Exclusion Criteria:

Acute promyelocytic leukemia with t(15;17)(q22;q12); or abnormal promyelocytic leukemia/retinoic acid receptor alpha (PML-RARA).
Participants with clinical symptoms suggestive of active central nervous system (CNS) leukemia or known CNS leukemia. Evaluation of cerebrospinal fluid is only required if there is a clinical suspicion of CNS involvement by leukemia during screening.
Participants with immediately life-threatening, severe complications of leukemia such as uncontrolled bleeding, pneumonia with hypoxia or shock, and/or disseminated intravascular coagulation.
Concomitant malignancy, requiring active treatment, except for basal-cell or squamous cell carcinoma of the skin, carcinoma-in-situ of the uterine cervix, or localized prostate cancer.
Adjuvant therapy for breast cancer or prostate cancer is allowed.
Active, uncontrolled, systemic infection or severe localized infection during screening or prior to Cycle 1 Day 1 (C1D1; unless considered due to tumor by the investigator).
Participants receiving prophylactic anti-infectives are allowed on study.
Known human immunodeficiency virus (HIV) infection with CD4+ T cell counts <350 cells/μL, initiation of antiretroviral therapy within 4 weeks before C1D1, or acquired immunodeficiency syndrome (AIDS)-related infection within 12 months before C1D1.
Hepatitis B virus (HBV) or hepatitis C virus (HCV) infection with viral load above the limit of quantification.
Major cardiac abnormalities as defined but not limited to the following: uncontrolled angina or unstable life-threatening arrhythmias, history of myocardial infarction within 12 weeks prior to Baseline, Class 3 or higher New York Heart Association (NYHA) congestive heart failure, or left ventricular ejection fraction (LVEF)<45% as measured by echocardiogram (ECHO) within 28 days of C1D1.
Persistent (3 consecutive ECGs performed ≥5 minutes apart) prolongation of the corrected QT interval by Fredericia's method (QTcF) to >480 msec.
Gastrointestinal conditions that may interfere with the absorption of orally-administered drugs including but not limited to short gut syndrome, gastroparesis, inflammatory bowel disease, or acute pancreatitis.

### Example 3: Human Dosing of Compound 1 and Pharmacokinetic Data

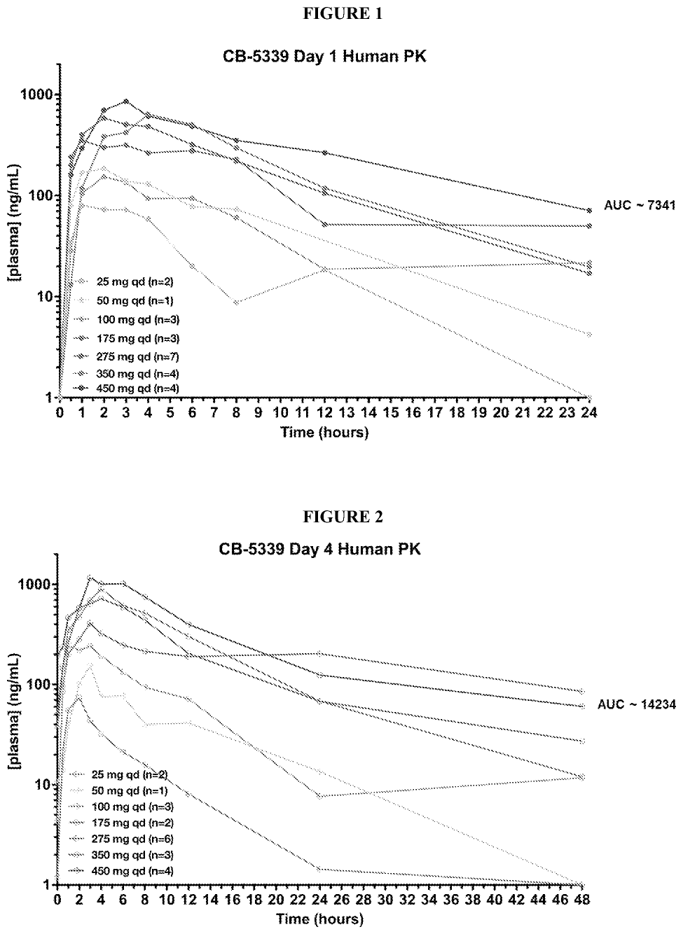

Subjects were treated with Compound 1 QD as described in Example 2. Plasma concentration of Compound 1 was assayed on day 1 at predosing and at 0.5, 1, 2, 4, 6, 8, 12 and 24 hours post-administration and on day 4 of dosing at predosing and at 0.5, 1, 2, 4, 6, 8, 12, 24 and 48 hours post-administration. Linear and semi-log plots of the plasma concentration-time plots are shown in FIGS. 1 and 2 for plasma concentration expressed as ng/mL and μM concentration units for dosing between 25 mg to 450 mg QD at day 1 (FIG. 1) and day 4 (FIG. 2). No adverse events were observed in any of the cohorts. No symptoms of visual impairment were seen with lower dose cohorts. Two patients in cohort X experienced low-grade visual symptoms on the day of dosing that did not persist. Table 3 shows exemplary_pharmacokinetic measurements analysis including Cmax, Cmax/dose, AUC (area under the curve), tmax, and half-life (t½) for two patients dosed in Cohort 1.

TABLE 3

| | Cₘₐₓ | Cₘₐₓ/Dose | AUC₀₋ₜ | AUC_{0-inf} | AUC_{0-inf}/Dose | tₘₐₓ | t_{1/2} | |
|---|---|---|---|---|---|---|---|---|
| Day | (ng/mL) | (ng/mL)/(mg) | (ng*hr/mL) | (ng*hr/mL) | (ng*hr/mL)/(mg) | (hr) | (hr) | AR |
| 1 | 81.7 | 3.27 | 364 | 425 | 17 | 2 | 2.61 | — |
| 4 | 74.3 | 2.97 | 337 | 367 | 14.7 | 2 | 3.84 | 0.914 |

| | Cmax | Cmax/Dose | AUC₀₋ₜ | AUC_{0-inf} | AUC_{0-inf}/Dose | | | |
|---|---|---|---|---|---|---|---|---|
| Day | (μM) | (μM)/(mg) | (μM*hr) | (μM*hr) | (μM*hr)/(mg) | | | |
| 1 | 0.198 | 0.00791 | 0.88 | 1.03 | 0.0412 | 2 | 2.61 | — |
| 4 | 0.18 | 0.00719 | 0.815 | 0.888 | 0.0355 | 2 | 3.84 | 0.914 |

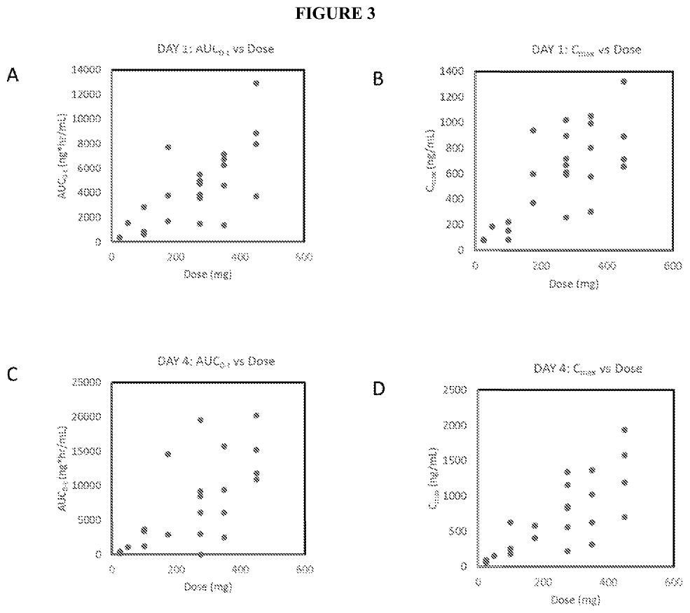

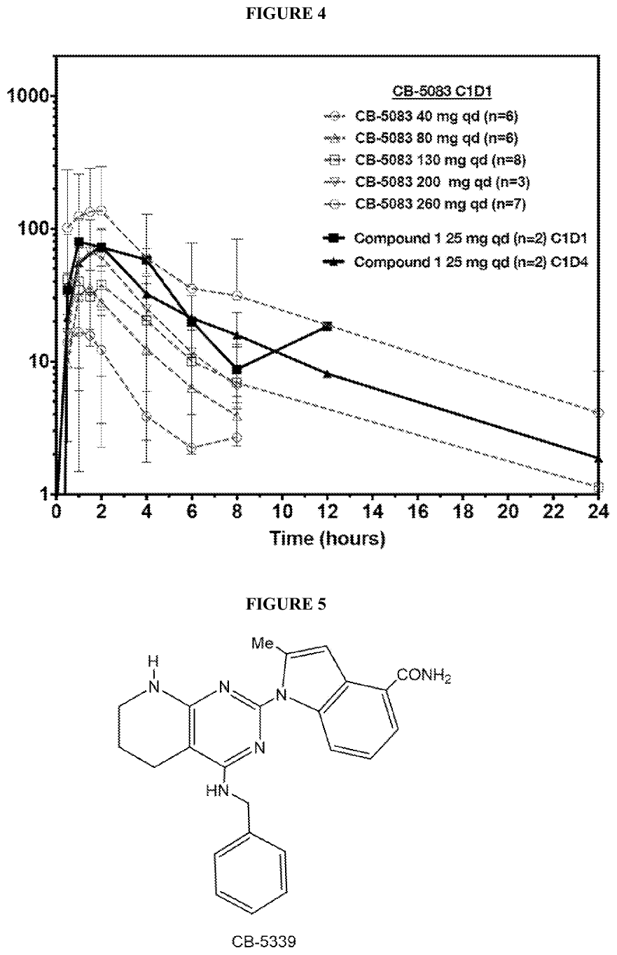

The results indicate a half-life of about 3-4 hours for Compound 1. No accumulation of Compound 1 was noted between days 1 and 4. Surprisingly, Compound 1 exhibited significantly higher plasma exposure than CB-5083 (1-[7,8-dihydro-4-[(phenylmethyl)amino]-5H-pyrano[4,3-d]pyrimidin-2-yl]-2-methyl-1H-indole-4-carboxamide), a compound tested previously in subjects. Comparatively Compound 1 at 25 mg QD produced significantly higher plasma exposure than CB-5083 administered at 40 mg QD. Exposures for Compound 1 were greater than dosing CB-5083 at greater than 200 mg. See FIG. 4. Also different from CB-5083, at equivalent plasma exposure Compound 1 produced no symptoms of visual impairment or other visual adverse events as seen with CB-5083 at the lower doses, and no lasting symptoms at the highest doses. As shown in FIGS. 1 and 2, exposure data from the treatment of patients dosed with 25 mg, 50 mg, 100 mg, 175 mg, 275 mg, 350 mg and 450 mg according to the study design in Example 2, the exposure level increases between the doses were generally proportional and linear. The AUC₀₋ₜ vs dose for Day 1 and Day 4 is shown in FIGS. 3A and 3C. The Cₘₐₓ vs dose is shown in FIGS. 3B and 3D for Day 1 and Day 4, respectively.

Two biomarkers were assessed in patients from cohorts 4 and 5. K48-Ub (ubiquitin) and Activated Caspase 3 biomarkers were analyzed from 5 patient blood samples. Two of the patients showed changes in the K48-Ub (ubiquitin) biomarker after treatment (best fold change approximately 3.5 fold). One of these patients also had a 9-fold increase in Activated Caspase 3.

### Example 4: Combinations of Compound 1 and Additional Therapeutic Agents

Following a similar protocol to Example 3, Compound 1 was tested with additional therapeutic agents. Each combination was tested in 3 cell lines: MOML-13 (Human Acute Myeloid Leukemia cell line; Addex Bio), MV-4-11 (Human B Myelomoncytic Leukemia cell line; ATCC), and OCI-AML-3 (Human Acute Myeloid Leukemia cell line). The genotype of these cell lines with respect to p53, FLT3-IDT, BRCA1 and BRCA2 is shown in Table 4.

TABLE 4

| | MOML-13 | MV-4-11 | OCI-AML-3 |
|---|---|---|---|
| p53 | WT | WT | WT |
| FLT3-IDT | HET | IDT | WT |
| BRCA1 | WT | WT | WT |
| BRCA-2 | MUT | WT | WT |

To test combinations of Compound 1 and the other therapeutic agents, concentration ranges for the dose responses were all set to evenly span a region 1 log higher to 1 log lower than the IC50 of the agent obtained in when each agent was used singly. Within this range, 20 dose levels were tested for each combination, with each response determined in duplicate. For each agent-cell line combination evaluated, the dose response of each individual drug was compared with dose responses of 3 combinations of the two agents, each mixed at three fixed ratios throughout the dose response: 1:1, 1:2, or 2:1, with Compound 1 always defined as the first agent of the ratios. The actual drug concentrations in the ratios were determined from the IC50 values obtained for each individual drug. For example, if Compound 1 IC50 was 1 uM and the additional agent IC50 was 10 uM, the actual ratio of the drugs for the 1:1 IC50 ratio is 1:10.

Raw data was analyzed using Graph Pad Prism, four parameter logistic fits, and Chou-Talalay drug combination analyses. The Chou-Talalay analysis requires that data be normalized and defined as the Fractional effect, which is a number required to be between 0 and 1, where 0 represents no effect (CTG signal in the absence of drug) and 1 represents the maximum possible effect (CTG signal in the absence of cells). In reality, many data points fell slightly outside these limits at the low or high end of the dose range. For data points with fractional effect >1, the Fe was set to 0.999. For data points with Fe<0, the fractional effect was set to 0.001. Data points with Fe>0.99 or <0.01 were omitted from the Chou-Talalay analysis because the Chou-Talalay analysis fits data to the Log of the fractional effect, with equal weight applied across the range of effect, such that the high and low end of the dose response, very small changes in effect within the range of experimental error erroneously skew the outcome of the model fit.

A Combination Index (CI) vs Fractional Effect plot showing the actual combination index plotted vs Fractional effect value was plotted for each combination dose. In this analysis, CI=1 represents additive activity, CI>1 represent less than additive activity (often referred to as antagonism) and CI<1=synergistic activity (better than additive). The CI scores across all 3 ratios were averaged and compared to Compound 1 alone. Results are shown in Table 5.

TABLE 5
Combination Index Values for Compound 1 with second
therapeutic agents tested in three cell lines

| | | Activity of Compound 1 and second | | |
|---|---|---|---|---|
| | | therapeutic agent in cell lines | | |
| MOA | Agent | MOML-13 | MV-4-11 | OCI-AML-3 |
| p53 | APR-246 | 1.228 | 1.285 | 1.279 |
| ATR | AZDO156 | 1.293 | 1.122 | 1.159 |
| ATM | AZD6738 | 1.338 | 1.228 | 1.421 |
| chemotherapeutic | cytarabine | 0.911 | 0.842 | 1.103 |
| FLT-3 | Gilteritinib | 1.266 | 0.799 | 1.514 |
| Proteasome | Ixazomib | 1.413 | 1.546 | 1.438 |
| DNA-PK | Nedisertib | 1.272 | 0.986 | 1.170 |
| NEDD-8 | Pevonedistat | 1.120 | 1.302 | 1.186 |
| PARP | Talazoparib | 0.529 | 0.769 | 0.986 |
| BH3 | Venetoclax | 0.730 | 0.819 | 0.855 |

A score between 0.200 to 0.800 demonstrates synergistic action between the combination; above 0.800 to 1.200 shows additive action and above 1.200 shows antagonistic effects of the combination. A number of the combinations demonstrated synergistic effects. The PARP inhibitor, Talazoparib showed strong synergy in combination with Compound 1, especially in the BRCA2 mutant cell line MOML-13. The FLT-3 agent Gilteritinib was synergistic in the homozygous mutant line, MV-4-11, and additive effects in the FLT3 wildtype and heterozygous AML lines. The combination of Compound 1 with Venetoclax showed synergy in the MOML-13 line and strong additive (near synergistic effects) in the other two AML lines.

Some agents such as the chemotherapeutic agent cytarabine produced additive effects across the cell lines. Others, such as the proteasome inhibitor ixazomib, were surprisingly antagonistic when placed in combination with Compound 1. Combination Index data with cytarabine, gilteritinib, talazoparib and venetoclax across ED50, ED75 and ED90 is shown below in Tables 6-9.

TABLE 6
Combination Index data for Compound 1 + Cytarabine

| Cell line | IC50 ratio | CI@ED50 | CI@ED75 | CI@ED90 |
|---|---|---|---|---|
| MOLM13 | 1:1 | 0.96199 | 0.86842 | 0.81297 |
| | 1:2 | 1.04698 | 0.94653 | 0.8963 |
| | 2:1 | 1.05439 | 0.87386 | 0.74172 |
| MV411 | 1:1 | 0.88589 | 1.01463 | 1.19702 |
| | 1:2 | 0.66123 | 0.65355 | 0.68017 |
| | 2:1 | 0.94868 | 0.81825 | 0.71705 |
| OCIAML3 | 1:1 | 1.10166 | 1.15893 | 1.25943 |
| | 1:2 | 1.07281 | 1.1129 | 1.18756 |
| | 2:1 | 1.03005 | 1.00189 | 1.00382 |

TABLE 7
Combination Index data for Compound 1 + Gilteritinib

| Cell line | IC50 ratio | CI@ED50 | CI@ED75 | CI@ED90 |
|---|---|---|---|---|
| MOLM13 | 1:1 | 1.36741 | 1.11099 | 1.30797 |
| | 1:2 | 1.45131 | 1.37816 | 1.31884 |
| | 2:1 | 1.09545 | 1.07758 | 1.07001 |
| MV411 | 1:1 | 1.0246 | 0.87599 | 0.79507 |
| | 1:2 | 0.84283 | 0.78281 | 0.77318 |
| | 2:1 | 0.8318 | 0.6791 | 0.58123 |
| OCIAML3 | 1:1 | 1.23768 | 1.41215 | 1.70347 |
| | 1:2 | 1.43917 | 1.61106 | 1.91545 |
| | 2:1 | 1.45767 | 1.41718 | 1.43636 |

TABLE 8
Combination Index data for Compound 1 + Talazoparib

| Cell line | IC50 ratio | CI@ED50 | CI@ED75 | CI@ED90 |
|---|---|---|---|---|
| MOLM13 | 1:1 | 0.52166 | 0.4849 | 0.56385 |
| | 1:2 | 0.55424 | 0.4023 | 0.42583 |
| | 2:1 | 0.66602 | 0.57757 | 0.56691 |
| MV411 | 1:1 | 0.7262 | 0.77595 | 0.85489 |
| | 1:2 | 0.62405 | 0.68188 | 0.78911 |
| | 2:1 | 0.75604 | 0.81539 | 0.89344 |
| OCIAML3 | 1:1 | 0.89558 | 0.85887 | 0.91508 |
| | 1:2 | 1.18857 | 1.03245 | 0.98467 |
| | 2:1 | 0.96909 | 0.96761 | 1.06213 |

TABLE 9
Combination Index data for Compound 1 + Venetoclax

| Cell line | IC50 ratio | CI@ED50 | CI@ED75 | CI@ED90 |
|---|---|---|---|---|
| MOLM13 | 1:1 | 0.82522 | 0.71944 | 0.6673 |
| | 1:2 | 0.72854 | 0.70503 | 0.74697 |
| | 2:1 | 0.87618 | 0.70898 | 0.59506 |
| MV411 | 1:1 | 0.76902 | 0.77942 | 0.84692 |
| | 1:2 | 0.89162 | 0.86077 | 0.90249 |
| | 2:1 | 0.7655 | 0.76198 | 0.79596 |
| OCIAML3 | 1:1 | 1.10939 | 0.68948 | 0.49606 |
| | 1:2 | 1.68801 | 0.92949 | 0.56929 |
| | 2:1 | 1.1625 | 0.63796 | 0.41303 |

### Example 5: Additional Combinations of Compound 1 and Therapeutic Agents

Compound 1 was tested in combination with additional therapeutic agents in the cell lines MOML-13, and MV-4-11 (see Example 4), and in HIL-60 (Human acute promyelocytic leukemia; ATCC). The additional therapeutic agents for this example were azacitidine (Cayman Chemical Cat No. 11164), decitabine (Sigma Cat No. A3656) and venetoclax (InvivoChem Cat. No. V0001).

Cell Culture: Three human acute myeloid leukemia tumor cell lines were used in this study: HL60, MV4;11 and MOLM-13. HL60 and MV4;11 were grown in IMDM base media with 20% and 10% FBS, respectively. MOLM-13 was grown in RPMI 1640 base media with 20% FBS. All culture media contained 2 mM glutamine, 100 units/mL sodium penicillin G, 25 g/mL gentamicin, and 100 g/mL streptomycin sulfate. The tumor cells were cultured in tissue culture flasks in a humidified incubator at 37° C., in an atmosphere of 5% CO2 and 95% air. Assay: Tumor cells were seeded in duplicate in a volume of 100 L/well of media into 96-well micro-culture plates at initial densities low enough to allow for several population doublings (typically three to five) during the indicated incubation period. After a 24 hour incubation, test agents were added to each well. After 72 hours of culture with test articles, cell viability was determined using the Cell Titer-Glo® (Promega G7571) assay. Briefly, Cell Titer-Glo® (Promega G7571) reagents were equilibrated at room temperature for 30 minutes; 100 μL per well was added at endpoint (72 hours). Plates were gently shaken for two minutes and then incubated at room temperature for ten minutes prior to reading luminescence on the BMG Clariostar Plus microplate reader.

For each drug agent on its own, IC₅₀ values in each cell line were determined and then used to select fixed drug concentrations near the IC₂₀ and IC₉₀ while varying Compound 1 concentrations in 20-point 1:1.5 dose-response curves, starting at 5 μM, and including a vehicle control (designated as “no fixed agent”). A second set of combinations consisted of fixed concentrations of Compound 1 near its IC₂₀ in each cell line or vehicle control, respectively, combined with varying concentrations of the second agent using a twenty-point dose-response of two-fold serial dilutions starting at 50 μM for Compound 1, 40 μM for azacitidine and decitabine, and 10 μM venetoclax.

Results are shown in Tables 10-12. An IC₅₀ ratio for each combination was calculated from the ratio of the combination IC50 as compared to the applicable no fixed agent (vehicle) control. Agents that exhibited enhanced activity in combination have ratios less than 1. For example, in MOML-13 cells, the combination of azacitidine and Compound 1 exhibited a ratio of 0.39 with both low and high doses of azacytidine in combination with Compound 1 (used as the variable dose agent), and a ratio of 0.28 when Compound 1 was provided as a fixed dose and azacitidine was provided across a range of doses. Combinations of Compound 1 with decitabine and with venetoclax+azacitidine also gave enhanced activity as compared to monotherapies. Similar enhanced activity with these combinations were also seen in MV4; 11 cells. These effects were much less pronounced in HL60 cells.

TABLE 10
Assessment of Combination Treatments in MOML-13 cell line

| | | | Combination:Monotherapy |
|---|---|---|---|
| Fixed Agent | Dose response Agent | IC₅₀ (nM) | IC₅₀ Ratio |
| No fixed agent (vehicle) | Compound 1 | 215.8 | |
| Azacitidine - 105 nM | Compound 1 | 84.6 | 0.39 |
| Azacitidine - 850 nM | Compound 1 | 84.9 | 0.39 |
| Decitabine - 2.5 nM | Compound 1 | 121.2 | 0.56 |
| Decitabine - 8.5 nM | Compound 1 | 65.0 | 0.30 |
| Venetoclax + Azacitidine - 6.5 nM | Compound 1 | 154.0 | 0.71 |
| Venetoclax + Azacitidine - 20 nM | Compound 1 | 112.7 | 0.52 |
| No fixed agent (vehicle) | Azacitidine | 444.8 | |
| Compound 1 - 75 nM | Azacitidine | 125 | 0.28 |
| No fixed agent (vehicle) | Decitabine | 10.8 | |
| Compound 1 - 75 nM | Decitabine | 6.5 | 0.60 |
| No fixed agent (vehicle) | Venetoclax + Azacitidine | 40.1 | |
| Compound 1 - 75 nM | Venetoclax + Azacitidine | 26.1 | 0.65 |

TABLE 11
Assessment of Combination Treatments in HL-60 cell line

| | | | Combination:Monotherapy |
|---|---|---|---|
| Fixed Agent | Dose response Agent | IC₅₀ (nM) | IC₅₀ Ratio |
| No fixed agent (vehicle) | Compound 1 | 305.3 | |
| Azacitidine - 5750 nM | Compound 1 | 310.6 | 1.02 |
| Azacitidine - 9800 nM | Compound 1 | 258.3 | 0.85 |
| Decitabine - 175 nM | Compound 1 | 293.8 | 0.96 |
| Decitabine - 1700 nM | Compound 1 | 322.5 | 1.06 |
| Venetoclax + Azacitidine - 85 nM | Compound 1 | 256.4 | 0.84 |
| Venetoclax + Azacitidine - 475 nM | Compound 1 | 255.9 | 0.84 |
| No fixed agent (vehicle) | Azacitidine | 2399.0 | |
| Compound 1 - 210 nM | Azacitidine | 1964.0 | 0.82 |
| No fixed agent (vehicle) | Decitabine | 5294.0 | |
| Compound 1 - 210 nM | Decitabine | 4031.0 | 0.76 |
| No fixed agent (vehicle) | Venetoclax + Azacitidine | 49.1 | |
| Compound 1 - 210 nM | Venetoclax + Azacitidine | 46.4 | 0.95 |

TABLE 12
Assessment of Combination Treatments in MV4; 11 cell line

| | | | Combination:Monotherapy |
|---|---|---|---|
| Fixed Agent | Dose response Agent | IC₅₀ (nM) | IC₅₀ Ratio |
| No fixed agent (vehicle) | Compound 1 | 251.1 | |
| Azacitidine - 500 nM | Compound 1 | 114.5 | 0.46 |
| Azacitidine - 3150 nM | Compound 1 | 118.0 | 0.47 |
| Decitabine - 30 nM | Compound 1 | 117.1 | 0.47 |
| Decitabine - 215 nM | Compound 1 | 91.5 | 0.36 |
| Venetoclax + Azacitidine - 20 nM | Compound 1 | 163.2 | 0.65 |
| Venetoclax + Azacitidine - 50 nM | Compound 1 | 118.4 | 0.47 |
| No fixed agent (vehicle) | Azacitidine | 1105.0 | |
| Compound 1 - 185 nM | Azacitidine | 109.5 | 0.10 |
| No fixed agent (vehicle) | Decitabine | 389.5 | |
| Compound 1 - 185 nM | Decitabine | 7.1 | 0.02 |
| No fixed agent (vehicle) | Venetoclax + Azacitidine | 53.2 | |
| Compound 1 - 185 nM | Venetoclax + Azacitidine | 18.2 | 0.34 |

Compound 1 was tested in combination with three PARP inhibitors in various cancer cell lines. The PARP inhibitors used for the study were talazoparib, olaparib and niraparib. Cisplatinin was used as a reference control.

TABLE 13
Cell lines used for study

| Cell line | Tumor type |
|---|---|
| HCC1395 | Breast |
| MX-1 | Breast |
| HCC1937 | Breast |
| HCC1806 | Breast |
| DU4475 | Breast |
| OVCAR-8 | Ovary |
| SK-OV-3 | Ovary |
| IGR-OV1 | Ovary |
| Capan-1 | Pancreas |
| DoTc2 4510 | Uterus/Cervix |

Cell lines were grown in media supplemented with 105% FBS, at a temperature of 37° C., 5% CO2 and 95% humidity, Following the protocol of Example 10, compounds and combinations were assayed on the cell lines using the Cell Titer-Glo® (Promega G7571) assay.

For each drug agent on its own, IC₅₀ values in each cell line were determined. IC50 values are shown in Table 14.

TABLE 14
IC50 values for single agents

| | Absolute IC50 (μM) | | | | |
|---|---|---|---|---|---|
| | Com- | | | | Cisplatin |
| Cell line | pound 1 | Niraparib | Olaparib | Talazoparib | (reference |
| HCC1395 | 0.201 | >5 | >5 | >5 | 4.75 |
| MX-1 | 0.409 | NA | >5 | NA | 6.60 |
| HCC1937 | 0.559 | NA | NA | >5 | 12.05 |
| HCC1806 | 0.437 | >5 | >5 | >5 | 3.24 |
| DU4475 | 0.343 | 1.02 | 4.52 | 0.03 | 1.60 |
| OVCAR-8 | 0.200 | >5 | >5 | >5 | 9.22 |
| SK-OV-3 | 1.792 | NA | NA | >5 | 8.30 |
| IGR-OV1 | 0.279 | >5 | >5 | NA | 1.45 |
| Capan-1 | 0.405 | >5 | >5 | 2.64 | 1.68 |
| DoTc2 4510 | 0.513 | NA | >5 | NA | 10.19 |

Interactions of the combinations of Compound 1 and each PARP inhibitor was assessed as follows: Each cell line was analyzed with Compound 1 alone and in matrix dose combinations with one of 3 PARP inhibitors in 72 hr Cell Titer Glow viability assays. Cisplatin and culture media were used as positive and negative control, respectively. The combination activities were assessed using CrownSyn software to determine synergy scores by Bliss Independence and Loewe additivity methods. Individual dose combination synergy scores over a value of 5 are synergistic with these methods and average synergy values give an idea of the synergy across all doses tested.

Synergy scores calculated by both Bliss independence model and Loewe additivity model (see Greco, W., Unkelbach, H. D., Poch, G., Suhnel, J., Kundi, M., & Bodeker, W. (1992). Consensus on concepts and terminology for combined-action assessment: the Saariselka agreement. Arch Complex Environ Stud, 4(3), 65-9). Without being bound by theory, the Bliss independence model is often applied to asses synergy for non-interacting drugs that elicit their responses independently, e.g., by targeting separate pathways, whereas the Loewe additivity model may be more compatible where both drugs have similar modes of action on the same targets or pathways. With these models, a score higher than 5 indicates synergy, and a score less than −5 indicates antagonism.

Table 15 shows the average Bliss score for the 6×6 matrix of each PARP inhibitor—Compound 1 combination tested across the cell lines, as well as the number of agent concentration combinations within the individual matrix with a Bliss score greater than 5. The results indicated that PARP inhibitor—Compound 1 combinations have synergistic effects in all cell lines tested for two of the PARP inhibitors and in all but two cell lines for the remaining PARP inhibitor, at specific dose concentrations.

TABLE 15
Compound 1 and PARP inhibitors on tumor cell lines

| | Talazoparib | | Olaparib | | Niraparib | |
|---|---|---|---|---|---|---|
| | Ave | # in matrix | Ave | # in matrix | Ave | # in matrix |
| Cell line | Bliss >5 | with Bliss >5 | Bliss >5 | with Bliss >5 | Bliss >5 | with Bliss >5 |
| SK-OV-3 | + | 13/36 | − | 10/36 | − | 8/36 |
| HCC1806 | − | 3/36 | − | 17/36 | − | 9/36 |
| MX-1 | − | 0/36 | − | 3/36 | − | 4/36 |
| IGR-OV1 | − | 7/36 | − | 12/36 | − | 7/36 |
| HCC1395 | − | 0/36 | − | 8/36 | − | 6/36 |
| OVCAR-8 | − | 6/36 | − | 8/36 | − | 9/36 |
| HCC1937 | − | 8/36 | + | 23/36 | − | 15/36 |
| DoTc2 4510 | − | 5/36 | − | 5/36 | − | 6/36 |
| Capan-1 | − | 4/36 | − | 4/36 | − | 2/36 |
| DU4475 | + | 15/36 | + | 13/36 | − | 10/36 |

### Example 6: Pharmacokinetic Analysis

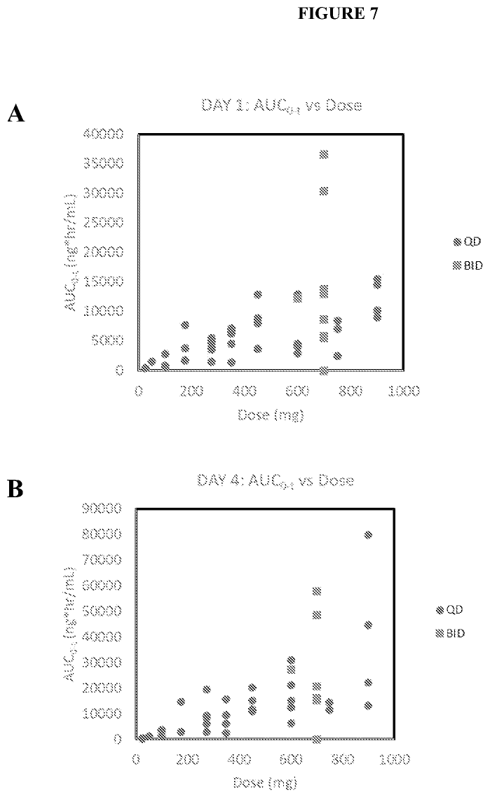

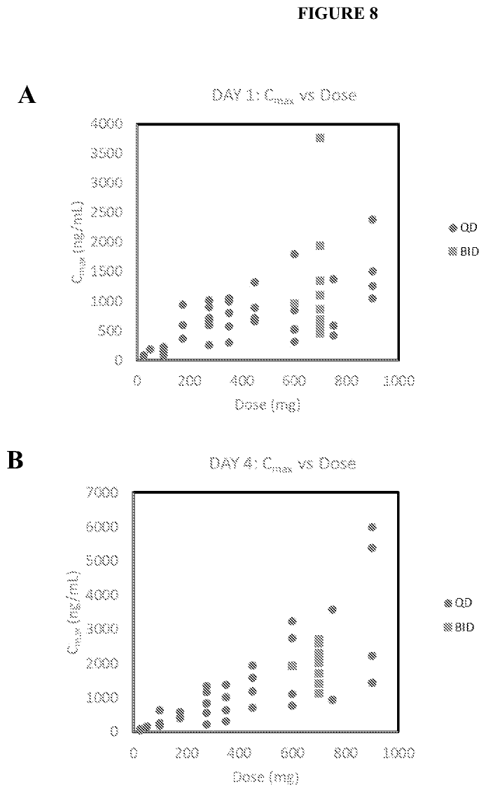

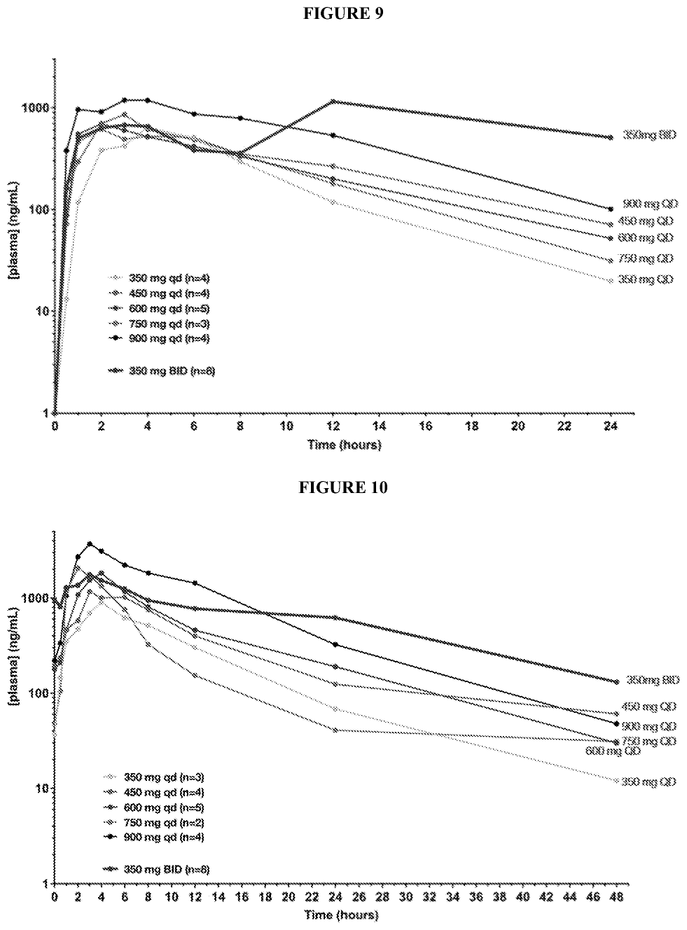

Subjects were treated with Compound 1 QD or BID dosing as described in Example 2 and blood samples were taken from subjects and analyzed as described in Example 3. Tables 16 and 17 show pharmacokinetic analyses for QD dosing for 100 mg, 175 mg, 275 mg, 350 mg, 400 mg and 900 mg cohorts and BID dosing for 300 mg and 350 mg cohorts. Abbreviations in Table 16 and Table 17: AR=Day 4 AUC₀₋₂₄/Day 1 AUC₀₋₂₄, NR=not reported; NA=not available. Plots of total daily dose versus AUC for QD and BID dosing are shown in FIGS. 7A and 7B for Day 1 and Day 4, respectively and plots of total daily dose versus Cmax for QD and BID dosing are shown in FIGS. 8A and 8B for Day 1 and Day 4, respectively. FIG. 9 and FIG. 10 show AUC for day 1 and day 4 of dosing with doses between 350-900 mg QD compared with 350 mg BID. Surprisingly, the BID dosing at the 350 mg dose gave a higher level of exposure on Day 1 than the exposure obtained with QD dosing at 600, 750 and 900 mg. The substantially increased exposure with BID dosing provides the benefit of being able to maintain elevated plasma levels for longer periods of time without raising the Cmax to overly high levels (thus potentially avoiding Cmax driven toxicity).

TABLE 16
Compound 1 Pharmacokinetics (ng/mL concentration units)

| | | | | | | | | | | |
|---|---|---|---|---|---|---|---|---|---|---|
| Dose | | | | | | | | | | |
| (mg)- | | Cₘₐₓ | Cₘₐₓ/Dose | AUC₀₋ₜ | AUC_{0-inf} | AUC_{0-inf}/Dose | tₘₐₓ | t_{1/2} | | # |
| Day | Values | (ng/mL) | (ng/mL)/(mg) | (ng*hr/mL) | (ng*hr/mL) | (ng*hr/mL)/(mg) | (hr) | (hr) | AR | subjects |

QD dosing

| | | | | | | | | | | |
|---|---|---|---|---|---|---|---|---|---|---|
| 100-1 | Mean | 154 | 1.54 | 1420 | 1780 | 17.8 | 2 | 6.87 | — | 3 |
| | SD | 70.3 | 0.703 | 1230 | 1830 | 18.3 | 0 | 4.68 | — | |
| 100-4 | Mean | 356 | 3.56 | 2750 | 3020 | 30.2 | 2.67 | 7.84 | 2.43 | 3 |
| | SD | 239 | 2.39 | 1330 | 1620 | 16.2 | 1.53 | 6.97 | 1.59 | |
| 175-1 | Mean | 636 | 3.63 | 4380 | 5260 | 30 | 3.33 | 11.4 | — | 3 |
| | SD | 284 | 1.62 | 3040 | 3460 | 19.8 | 2.52 | 10.4 | — | |
| 175-4 | Mean | 490 | 2.8 | 8760 | 2950 | 16.8 | 13.5 | 6.01 | 1.35 | 3 |
| | SD | NA | NA | NA | NA | NA | NA | NA | NA | |
| 275-1 | Mean | 679 | 2.47 | 4140 | 4660 | 16.9 | 2.57 | 5.08 | — | 7 |
| | SD | 243 | 0.885 | 1350 | 1600 | 5.82 | 1.13 | 1.22 | — | |
| 275-4 | Mean | 827 | 3.01 | 9230 | 9660 | 35.1 | 3.6 | 8.9 | 1.99 | 7 |
| | SD | 403 | 1.47 | 6220 | 6470 | 23.5 | 0.894 | 2.84 | 0.865 | |
| 350-1 | Mean | 746 | 2.13 | 5200 | 5330 | 15.2 | 3.2 | 4.48 | — | 5 |
| | SD | 309 | 0.882 | 2370 | 2400 | 6.86 | 1.1 | 0.732 | — | |
| 350-4 | Mean | 834 | 2.38 | 8440 | 8570 | 24.5 | 4 | 7.55 | 1.38 | 4 |
| | SD | 458 | 1.31 | 5610 | 5740 | 16.4 | 0 | 2.06 | 0.673 | |
| 450-1 | Mean | 895 | 1.99 | 8340 | 8860 | 19.7 | 3 | 6.05 | — | 4 |
| | SD | 301 | 0.668 | 3770 | 5230 | 11.6 | 0.816 | 1.76 | — | |
| 450-4 | Mean | 1350 | 3.01 | 14500 | 15900 | 35.2 | 4 | 9.62 | 1.81 | 4 |
| | SD | 530 | 1.18 | 4200 | 3400 | 7.55 | 1.41 | 5.02 | 0.983 | |
| 600-1 | Mean | 799 | 1.33 | 5710 | 6500 | 10.1 | 3.6 | 5.94 | — | 5 |
| | SD | 590 | 0.984 | 4060 | 4920 | 7.31 | 2.61 | 1.25 | — | |
| 600-4 | Mean | 1960 | 3.27 | 17200 | 18200 | 30.3 | 3.4 | 9.77 | 2.68 | 5 |
| | SD | 1050 | 1.74 | 9350 | 9460 | 15.8 | 0.894 | 2.38 | 0.589 | |
| 750-1 | Mean | 796 | 1.06 | 5960 | 6160 | 8.21 | 3.33 | 4.49 | — | 3 |
| | SD | 512 | 0.68 | 3100 | 3140 | 4.18 | 2.31 | 0.374 | — | |
| 750-4 | Mean | 2260 | 3.02 | 12900 | 13200 | 17.6 | 2.5 | 6.67 | 2.96 | 2 |
| 900-1 | Mean | 1550 | 1.73 | 12300 | 14000 | 14.5 | 2.5 | 5.71 | — | 4 |
| | SD | 589 | 0.66 | 3170 | 4220 | 4.36 | 1.29 | 2.05 | — | |
| 900-4 | Mean | 3220 | 3.58 | 26700 | 27000 | 30 | 3.33 | 6.47 | 1.79 | 4 |
| | SD | 2430 | 2.70 | 16200 | 16500 | 18.3 | 0.577 | 1.11 | 0.776 | |

BID dosing

| | | | | | | | | | | |
|---|---|---|---|---|---|---|---|---|---|---|
| 300-1 | — | 965 | 1.61 | 12200 | NA | NA | 12 | NA | — | 1 |
| 300-4 | — | 1920 | 3.2 | 27400 | 27700 | 46.1 | 3 | NR | 1.72 | 1 |
| 350-1 | Mean | 1340 | 1.92 | 14600 | 29600 | 42.4 | 7.63 | NR | — | 9 |
| | SD | 1090 | 1.56 | 12300 | 13500 | 19.3 | 7.91 | NR | — | |
| 350-4 | Mean | 2020 | 2.89 | 23400 | 31800 | 45.4 | 3.25 | NR | 2.66 | 9 |
| | SD | 556 | 0.794 | 18900 | 20700 | 29.6 | 3.85 | NR | 1.48 | |

-

TABLE 17
Compound 1 Pharmacokinetics (μM concentration units)

| | | | | | | | | | | |
|---|---|---|---|---|---|---|---|---|---|---|
| Dose | | | | | | | | | | |
| (mg)- | | Cmax | Cmax/Dose | AUC₀₋ₜ | AUC_{0-inf} | AUC_{0-inf}/Dose | | | | # |
| Day | Values | (μM) | (μM)/(mg) | (μM*hr) | (μM*hr) | (μM*hr)/(mg) | tₘₐₓ | t_{1/2} | AR | subjects |

QD dosing

| | | | | | | | | | | |
|---|---|---|---|---|---|---|---|---|---|---|
| 100-1 | Mean | 0.372 | 0.00372 | 3.43 | 4.31 | 0.0431 | 2 | 6.87 | — | 3 |
| | SD | 0.17 | 0.0017 | 2.97 | 4.44 | 0.0444 | 0 | 4.68 | — | |
| 100-4 | Mean | 0.861 | 0.00861 | 6.66 | 7.32 | 0.0732 | 2.67 | 7.84 | 2.43 | 3 |
| | SD | 0.578 | 0.00578 | 3.22 | 3.92 | 0.0392 | 1.53 | 6.97 | 1.59 | |
| 175-1 | Mean | 1.54 | 0.0088 | 10.6 | 12.7 | 0.0727 | 3.33 | 11.4 | — | 3 |
| | SD | 0.688 | 0.00393 | 7.37 | 8.37 | 0.0478 | 2.52 | 10.4 | — | |
| 175-4 | Mean | 1.19 | 0.00678 | 21.2 | 7.13 | 0.0408 | 13.5 | 6.01 | 1.35 | 3 |
| | SD | NA | NA | NA | NA | NA | NA | NA | NA | |
| 275-1 | Mean | 1.65 | 0.00598 | 10 | 11.3 | 0.041 | 2.57 | 5.08 | — | 7 |
| | SD | 0.59 | 0.00214 | 3.26 | 3.88 | 0.0141 | 1.13 | 1.22 | — | |
| 275-4 | Mean | 2.00 | 0.00728 | 22.4 | 23.4 | 0.085 | 3.6 | 8.9 | 1.99 | 7 |
| | SD | 0.976 | 0.00355 | 15.1 | 15.7 | 0.0569 | 0.894 | 2.84 | 0.865 | |
| 350-1 | Mean | 1.81 | 0.00516 | 12.6 | 12.9 | 0.0369 | 3.2 | 4.48 | — | 5 |
| | SD | 0.747 | 0.00213 | 5.73 | 5.81 | 0.0166 | 1.1 | 0.732 | — | |
| 350-4 | Mean | 2.02 | 0.00577 | 20.4 | 20.8 | 0.0593 | 4 | 7.55 | 1.38 | 4 |
| | SD | 1.11 | 0.00317 | 13.6 | 13.9 | 0.0397 | 0 | 2.06 | 0.673 | |
| 450-1 | Mean | 2.17 | 0.00481 | 20.2 | 21.5 | 0.0477 | 3 | 6.05 | — | 4 |
| | SD | 0.728 | 0.00162 | 9.12 | 12.7 | 0.0282 | 0.816 | 1.76 | — | |
| 450-4 | Mean | 3.28 | 0.00728 | 35.2 | 38.4 | 0.0853 | 4 | 9.62 | 1.81 | 4 |
| | SD | 1.28 | 0.00285 | 10.2 | 8.23 | 0.0183 | 1.41 | 5.02 | 0.983 | |
| 600-1 | Mean | 1.93 | 0.00322 | 13.8 | 15.7 | 0.0244 | 3.6 | 5.94 | — | 5 |
| | SD | 1.43 | 0.00238 | 9.82 | 11.9 | 0.0177 | 2.61 | 1.25 | — | |
| 600-4 | Mean | 4.75 | 0.00792 | 41.7 | 44 | 0.0733 | 3.4 | 9.77 | 2.68 | 5 |
| | SD | 2.54 | 0.00423 | 22.6 | 22.9 | 0.0382 | 0.894 | 2.38 | 0.589 | |
| 750-1 | Mean | 1.93 | 0.00257 | 14.4 | 14.9 | 0.0199 | 3.33 | 4.49 | — | 3 |
| | SD | 1.24 | 0.00165 | 7.5 | 7.6 | 0.0101 | 2.31 | 0.374 | — | |
| 750-4 | Mean | 5.48 | 0.00731 | 31.3 | 32.1 | 0.0427 | 2.5 | 6.67 | 2.96 | 3 |
| 900-1 | Mean | 3.76 | 0.00418 | 29.8 | 33.9 | 0.0351 | 2.5 | 5.71 | — | 4 |
| | SD | 1.43 | 0.00159 | 7.69 | 10.2 | 0.0106 | 1.29 | 2.05 | — | |
| 900-4 | Mean | 7.8 | 0.00866 | 64.6 | 65.3 | 0.0726 | 3.33 | 6.47 | 1.79 | 4 |
| | SD | 5.89 | 0.01 | 39.3 | 39.9 | 0.0444 | 0.577 | 1.11 | 0.776 | |

BID dosing

| | | | | | | | | | | |
|---|---|---|---|---|---|---|---|---|---|---|
| 300-1 | — | 2.34 | 0.00389 | 29.6 | NA | NA | 12 | NA | — | 1 |
| 300-4 | — | 4.65 | 0.00775 | 66.4 | 67 | 0.112 | 3 | NR | 1.72 | 1 |
| 350-1 | Mean | 3.25 | 0.00464 | 35.3 | 71.8 | 0.103 | 7.63 | NR | — | 9 |
| | SD | 2.64 | 0.00377 | 29.9 | 32.8 | 0.0468 | 7.91 | NR | — | |
| 350-4 | Mean | 4.89 | 0.00699 | 56.7 | 77 | 0.11 | 3.25 | NR | 2.66 | 9 |
| | SD | 1.35 | 0.00192 | 45.9 | 50.2 | 0.0717 | 3.85 | NR | 1.48 | |

We claim:

1. A method of treating cancer in a subject in need thereof comprising administering to the subject a pharmaceutical composition comprising 1-(4-(benzylamino)-5,6,7,8-tetrahydropyrido[2,3-d]pyrimidin-2-yl)-2-methyl-1H-indole-4-carboxamide, or a pharmaceutically acceptable salt thereof, at a dose over a 24-hour period of about 450 mg to about 1500 mg, whereby the subject experiences a therapeutic response.

2. The method of claim 1, wherein the pharmaceutical composition comprises a tosylate salt of 1-(4-(benzylamino)-5,6,7,8-tetrahydropyrido[2,3-d]pyrimidin-2-yl)-2-methyl-1H-indole-4-carboxamide.

3. The method of claim 1, wherein the dose over a 24-hour period is between 450 mg to about 1250 mg, about 450 mg to about 1000 mg, about 450 mg to about 900 mg, about 450 mg to about 850 mg, about 450 mg to about 800 mg, about 450 mg to about 750 mg, about 450 mg to about 700 mg, about 450 mg to about 650 mg, about 450 mg to about 600 mg, about 600 mg to about 1250 mg, about 600 mg to about 1000 mg, about 600 mg to about 900 mg, about 600 mg to about 850 mg, about 600 mg to about 800 mg, about 600 mg to about 750 mg, about 600 mg to about 700 mg, about 900 mg to about 950 mg, about 900 mg to about 1000 mg, about 900 mg to about 1250 mg, about 1000 mg to about 1250 mg, about 1000 mg to about 1500 mg, or about 1250 mg to about 1500 mg.

4. The method of claim 1, wherein the dose administered over the 24-hour period is about 450 mg, 500 mg, 550 mg, 600 mg, 650 mg, 700 mg, 750 mg, 800 mg, 850 mg, 900 mg, 950 mg, 1000 mg, 1250 mg or 1500 mg.

5. The method according to any of claims 1-4, wherein the cancer is a hematological cancer.

6. The method of claim 5, wherein the cancer is selected from the group consisting of acute myeloid leukemia (AML), myelodysplastic syndrome (MDS), myelodysplastic/myeloproliferative overlap neoplasms (MDS/MPN), CMML (chronic myelomonocytic leukemia), atypical chronic myeloid leukemia (aCML), multiple myeloma, myeloma, amyloidosis, Waldenstrom's macroglobulinemia (also known as lymphoplasmacytic lymphoma), acute lymphoblastic leukemia (ALL), B-lymphoblastic leukemia, T-lymphoblastic leukemia, lymphoma, B-cell acute lymphoblastic leukemia, T-cell acute lymphoblastic leukemia, B-cell acute lymphoblastic lymphoma, T-cell acute lymphoblastic lymphoma, Burkitt's leukemia/lymphoma, Non-Hodgkin's lymphoma (NHL), chronic lymphocytic leukemia (CLL), small lymphocytic lymphoma (SLL), B-cell NHL, follicular lymphoma, marginal zone lymphoma, mantle cell lymphoma, diffuse large B-cell lymphoma (DLBCL), double/triple hit B-cell lymphoma, myeloproliferative neoplasm (MPN), essential thrombocythemia (ET), polycythemia vera (PV), myelofibrosis, primary myelofibrosis, post-PV myelofibrosis, Post-ET myelofibrosis, chronic myeloid leukemia (CML), blastic plasmacytoid dendritic cell neoplasm (BPDCN), M3 AML, and APL (acute promyelocytic leukemia).

7. The method according to any of claims 1-4, wherein the cancer is acute myeloid leukemia (AML) or myelodysplastic syndrome (MDS).

8. The method of claim 7, wherein the AML is relapsed AML, recurrent AML, refractory AML, or any combination thereof.

9. The method of claim 7, wherein the AML is de novo AML, secondary AML including therapy related AML and AML with myelodysplasia-related changes (AML with MRC), biphenotypic acute leukemia (also referred to as acute leukemia of ambiguous lineage), or AML with recurrent abnormalities.

10. The method of claim 7, wherein the AML is AML with an actionable mutation.

11. The method of claim 7, wherein the AML is AML without an actionable mutation.

12. The method of claim 7, wherein the MDS is relapsed or refractory MDS.

13. The method of claim 7, wherein the MDS is classified by the Revised International Prognostic Scoring System (IPSS-R) as low risk MDS, intermediate risk MDS, high risk MDS, or very high risk MDS.

14. The method of claim 7, wherein the MDS is selected from the group consisting of MDS with single lineage dysplasia (MDS-SLD), MDS with multilineage dysplasia (MDS-HLD), MDS with ringed sideroblasts (MDS-RS), MDS with Ringed Sideroblasts with single lineage dysplasia (MDS-RS-SLD), MDS with Ringed Sideroblasts (MDS-RS), MDS with Ringed Sideroblasts with multilineage dysplasia (MDS-RS-MLD), MDS with excess blasts 1 and/or 2 (MDS-EB-1, MDS-EB-2), MDS unclassifiable (MDS-U), and MDS with isolated del (5q).

15. The method according to any of claims 7-14, wherein the subject is treated irrespective of the subject's mutation or cytogenetic status.

16. The method according to any of claims 5-14, wherein therapeutic response comprises a complete remission, complete remission without minimal residual disease, complete remission with incomplete hematologic recovery, morphologic leukemia-free state or partial remission, hematological improvement, complete cytogenetic response, transfusion independence, red blood cell transfusion independence or platelet transfusion independence, or eligibility for stem cell transplantation.

17. The method according to any of claims 1-14, wherein therapeutic response comprises an increase in overall survival, an increase in relapse free survival, an increase in event free survival, an increased duration of response or a reduction in cumulative incidence of relapse.

18. The method according to any of claims 1-17, wherein the pharmaceutical composition is administered in a regimen comprising (a) 4 sequential days of administering the drug to a subject followed by 3 sequential days of no administration, (b) 5 sequential days of administering the drug to a subject followed by 2 sequential days of no administration, (c) a once weekly dosage, or (d) a twice-weekly dosage.

19. The method of claim 18, wherein the administration regimen is repeated.

20. The method of claim 18, wherein the pharmaceutical composition is administered in a 28 day cycle comprising administration on days 1-4, 8-11, 15-18, 22-25 of each cycle.

21. The method of claim 20, wherein the 28 day cycle is repeated at least once.

22. The method according to any of claims 1-21, wherein the pharmaceutical composition is administered once daily on the days of administration.

23. The method according to any of claims 1-21, wherein the pharmaceutical composition is administered two times per day on the days of administration.

24. The method according to any of claims 1-21, wherein the dose over a twenty-four hour period is administered as a twice daily dose on the days of administration, and wherein the total dose is divided into two lesser doses.

25. The method according to claim 24, wherein the two lesser doses each contain the same dose.

26. The method according to claim 24, wherein the two lesser doses each contain a different dose.

27. The method according to any of claims 24-26, wherein the dose over a twenty-four hour period is administered as a twice daily dose on the days of administration, wherein the time interval between the administration of the first daily dose and the administration of the second daily dose is 8 to 12 hours.

28. The method according to any of claims 1-27, wherein the pharmaceutical composition is administered orally.

29. The method according to any of claims 1-28, wherein the pharmaceutical composition is administered as a tablet or a capsule.

30. The method according to any of claims 7-29, wherein the cancer is AML and the subject carries one or more mutations in a locus selected from the group consisting of ABL1, ASXL1, BCOR, BCORL1, BCR, BRAF, CALR, CBFB, CBL, CBLB, CDKN2A, CEBPA, CSF3R, CUX1, DEK, DNMT3A, ETV6, EZH2, FBXW7, FLT3, GATA1, GATA2, GNAS, HRAS, IDH1, IDH2, IKZF1, JAK2, JAK3, KDM6A, KIT, KMT2A, MECOM (EVI1), MLL, MLLT3, MPL, MYD88, MYH11, NOTCH1, NPM1, NUP214, NRAS, PDGFRA, PHF6, PTEN, PTPN11, RAD21, RUNX1, SF3B1, SRSF2, SMC1A, SMC3, STAG2, TET2, TP53, U2AF1, WT1, and ZRSR2.

31. The method according to any of claims 1-30, wherein the treatment further includes administration of a second therapeutic agent.

32. The method of claim 31, wherein the second therapeutic agent is a DNA damaging agent, a hypomethylating agent, an agent that interferes with DNA synthesis or an agent that interferes with DNA replication.

33. The method of claim 32, wherein the second therapeutic agent is decitabine, azacytidine, or cytarabine.

34. The method of claim 33, wherein the second therapeutic agent is cytarabine dosed in a 7+3 regimen with an anthracycline antibiotic.

35. The method of claim 34, the 7+3 comprises 7 days of cytarabine and 3 days of an anthracycline antibiotic selected from daunorubicin, doxorubicin, idarubicin, and mitoxantrone.

36. The method of claim 31, wherein the second therapeutic agent is a tyrosine kinase inhibitor.

37. The method of claim 31, wherein the second therapeutic agent is a DNA damage repair inhibitor.

38. The method of claim 37, wherein the second therapeutic agent is an inhibitor of ATM, ATR, PARP, or Chk1.

39. The method of claim 31, wherein the second therapeutic agent is a proteasome inhibitor.

40. The method of claim 39, wherein the second therapeutic agent is Velcade (bortezomib), Ninlaro (ixazomib) or Kyprolis (carfilzomib).

41. The method of claim 39, wherein the second therapeutic agent is lenalidomide, pomalidomide, dexamethasone or a combination thereof.

42. The method of claim 31, wherein the second therapeutic agent is an inhibitor of FLT3, IDH1, or IDH2.

43. The method of claim 31, wherein the second therapeutic agent is an immune oncology agent or an immune modulation agent.

44. The method according to any of claims 1-4, 17-29, 31-43, wherein the cancer is selected from the group consisting of a solid tumor, a metastatic form of a solid tumor, an advanced metastatic solid tumor, a lymphoma and an advanced lymphoma.

45. The method of claim 44, wherein the subject has undergone at least one prior therapy.

46. The method of claim 42, wherein the second therapeutic agent comprises gilteritinib or an analog thereof.

47. The method of claim 42, wherein the second therapeutic agent comprises midostaurin or an analog thereof.

48. The method of claim 46 or claim 47, wherein the cancer comprises a FLT3 mutation.

49. The method of claim 31, wherein the second therapeutic agent inhibits poly ADP ribose polymerase (PARP).

50. The method of claim 49, wherein the second therapeutic agent comprises talazoparib, olaparib, niraparib, or an analog thereof.

51. The method of claim 49 or claim 50, wherein the cancer comprises a BRCA-2 mutation.

52. The method of claim 49 or claim 50, wherein the cancer comprises a mutation that impairs homologous recombination.

53. The method of claim 31, wherein the second therapeutic agent inhibits Bcl-2.

54. The method of claim 53, wherein the second therapeutic agent comprises a BH3 mimetic.

55. The method of claim 54, wherein the BH3 mimetic comprises venetoclax or an analog thereof.

56. The method of claim 55, wherein the method further comprises the administration of a third therapeutic agent.

57. The method of claim 56, wherein the third therapeutic agent comprises azacitidine.

58. The method of claim 31, wherein the second therapeutic agent comprises an anti-CD38 antibody.

59. The method of claim 58, wherein the anti-CD38 antibody is Sarclisa (isatuximab).

60. The method of claim 58, wherein the anti-CD38 antibody is daratumumab.

61. The method of claim 31, wherein the second therapeutic agent comprises a menin inhibitor.

62. The method of claim 61, wherein the second therapeutic agent comprises SNDX-5613.

63. The method of claim 61, wherein the second therapeutic agent comprises KO-539.

64. The method of claim 31, wherein the second therapeutic agent comprises a selective inhibitor of nuclear export.

65. The method of claim 64, wherein the second therapeutic agent comprises selinexor.

66. The method of claim 5, wherein the cancer is a bcr-abl negative myeloid neoplasm.

67. The method according to any of claims 1-66, wherein the administration does not result in a visual impairment of the subject.

68. The method of any one of claims 27-65, and 67, wherein the second therapeutic agent is administered prior to the administration of the pharmaceutical composition.

69. The method of claim 68, wherein the second therapeutic agent is administered at about 24 hours or 1 day prior to the administration of the pharmaceutical composition.
